(19) United States
(12) Patent Application Publication
(10) Pub. No.: US 2003/0165479 A1
(43) Pub. Date: Sep. 4, 2003

(54) Methods for isolating proteins expressed by dendritic cells

(76) Inventors: Mark A. Velleca, New Haven, CT (US); Ira Mellman, Guilford, CT (US)

Correspondence Address: MORGAN LEWIS & BOCKIUS LLP, 1111 PENNSYLVANIA AVENUE NW, WASHINGTON, DC, 20004, US

(21) Appl. No.: 10/100,082

(22) Filed: Mar. 19, 2002

Related U.S. Application Data
(60) Provisional application No. 60/276,474, filed on Mar. 19, 2001.

(51) Int. Cl. C12P 21/02; A61K 48/00; C12N 5/08; A61K 38/19

(52) U.S. Cl. 424/93.21; 424/85.1; 435/69.1; 435/69.5; 435/372; 435/320.1

(57) ABSTRACT

Disclosed are methods for generating dendritic cells and isolating dendritic cell associated proteins. Also disclosed are compositions and methods for generating an antibody against a dendritic cell associated protein as well as methods for modulating the expression or activity of dendritic cell associated proteins. The compositions and methods of the invention are useful in the diagnosis and treatment of diseases associated with altered dendritic cell activity.

### FIELD OF THE INVENTION

The invention relates to methods of generating mature dendritic cells. The invention also relates to methods of identifying proteins or polypeptides selectively expressed by mature dendritic cells.

### BACKGROUND OF THE INVENTION

Dendritic cells are potent antigen presenting cells responsible for inducing antigen-specific immunity. Although the functions and clinical utility of dendritic cells have thus become topics of great interest, fundamental questions remain concerning their origin, regulation and activities. Multiple populations of dendritic cells exist that are derived from various lineages, take up residence in different tissues, and have distinct functional attributes. In addition, dendritic cells exhibit a pattern of terminal differentiation resulting in their conversion from immature cells specialized for antigen accumulation to mature cells specialized for T cell stimulation.

Dendritic cell maturation can be triggered by a variety of cytokines in vitro (e.g., TNF-alpha) and bacterial products (e.g., LPS). Maturation in vivo has best been described for epidermal dendritic cells (Langerhans cells), an immunologically important dendritic cell population. Langerhans cells exist as immature cells in the skin that migrate into the afferent lymphatics and then to lymphoid tissue. Migration is enhanced by the presence of maturational stimuli, as would occur concomitant with infection. However, movement of Langerhans cells to lymph nodes also occurs constitutively, with maturation subsequently induced in lymphoid tissue upon binding of CD40 on Langerhans cells to CD40 ligand (CD40L) on T cells. There is increasing evidence that the control of Langerhans cell maturation may help determine the induction of tolerogenic versus immunogenic T cell responses. However, progress in understanding Langerhans cell maturation at the molecular level has been limited by the inability to produce immature Langerhans cells in culture. Although the maturation of human monocyte- and mouse bone marrow-derived dendritic cells have begun to be evaluated in some detail, Langerhans cells have proved difficult to isolate in any quantity, purity or defined maturational stage starting from bone marrow aspirates, leukapheresis products, or umbilical cord blood. Thus, little is known regarding the maturation of this important dendritic cell population.

By using CD34⁺ cells from G-CSF-mobilized patients, the inventors have been able to produce large numbers of Langerhans cells that can be maintained in an immature state and induced to mature synchronously by the addition of inflammatory mediators. Remarkably, the type of activation stimulus used herein controls the type of mature dendritic cell that emerges, both in terms of overall cellular organization, cytokine production, and expression of receptors that play critical roles in innate immunity. It is becoming apparent that dendritic cells are not a single, homogeneous cell type, but rather a system of cells of multiple lineages and activation states. Although different dendritic cell populations are becoming associated with different immunological outcomes, the biological basis for these differences remains poorly understood. In humans, only dendritic cells derived from peripheral blood monocytes grown in GM-CSF and IL-4 have been studied in any detail. Despite several attempts, it has thus far proved difficult to establish an analogous system to study the differentiation and early stages of maturation of human dendritic cells all the way from CD34⁺ precursors.

Several groups have differentiated dendritic cells in vitro, even dendritic cells of the Langerhans cell type from CD34⁺ cells isolated from cord blood. However, previous approaches yielded relatively small numbers of heterogeneous cells that invariably exhibited a high level of MHC II expression by the end of the isolation procedure, precluding the possibility of analyzing events related to maturation. The inventors have developed methods that permit the generation of larger numbers of virtually homogeneous dendritic cells from CD34⁺ cells isolated from apheresis products.

### SUMMARY OF THE INVENTION

In one embodiment the invention encompasses a method for selective production of mature human dendritic cell subtypes comprising generating immature human dendritic cells expressing CD1a antigen; and culturing the immature human dendritic cells in a medium comprising an agent selected from the group consisting of TNF-alpha, LPS and CD40 ligand. In one embodiment, the agent selected determines the subtype of dendritic cell produced. In some embodiments, the immature human dendritic cells are Langerhans cells. According to the methods of the invention the concentration of TNF-alpha is about 1.0 to 4.0 units/ml and LPS about 200 to 300 ng/ml. The CD40 ligand employed in this method can be membrane bound on platelets which are co-cultured with the immature human dendritic cells. The medium can further comprise one or more agents selected from the group consisting of GM-CSF, SCF, TGF-beta-1 and Flt3 ligand.

The immature human dendritic cells can be generated by a method comprising immunopurifying CD34 antigen expressing cells from a human blood sample from an individual treated with G-CSF; culturing the CD34 antigen expressing cells in a medium comprising GM-CSF, SCF, TNF-alpha, TGF-beta-1 and/or Flt3 ligand until clusters of non-adherent cells form; isolating the non-adherent cluster cells; and further culturing the isolated non-adherent cluster cells in a medium comprising GM-CSF, SCF, TNF-alpha, TGF-beta-1 and/or Flt3 ligand. The concentration of GM-CSF in the medium is about 2.5×10⁻⁴ to 7.5×10⁻⁴ units/ml while the concentration of SCF is about 0.5 to 2.0 units/ml and the concentration of TNF-alpha is about 0.25 to 1.0 units/ml. The concentration of TGF-beta-1 is about 0.05 to 0.2 units/ml while the concentration of Flt3 ligand is about 50 to 150 ng/ml.

In another embodiment, the invention encompasses a method of isolating a dendritic cell associated protein comprising treating a dendritic cell with a first agent which alters the expression or activity of a protein associated with the dendritic cell; isolating a cell extract from the dendritic cell; associating the extract with at least one second agent; detecting the binding of the second agent to the dendritic cell associated protein in the extract; and isolating the dendritic cell associated protein bound to the second agent from the extract. Alternatively, instead of the first agent being contacted with the dendritic cell, mechanical disruption of a dendritic cell can be used.

In some embodiments, the first agent is selected from the group consisting of proteins and nucleic acids. Preferably, the nucleic acid is selected from the group consisting of double stranded RNA and CpG DNA. Preferably, the protein is a human protein, more preferably, a cytokine protein, while in another embodiment the protein is a pathogen isolated from a microorganism selected from the group consisting of viruses, bacteria, fungi and parasites. In other embodiments, the protein is selected from the group consisting of interleukin-1beta, prostaglandin-E2, tumor necrosis factor-alpha, lipopolysaccharide and CD40 ligand.

In some embodiments of the methods of the invention, the cell extract is isolated from a cell component selected from the group consisting of the nucleus, cytosol and cell membrane. In one embodiment the dendritic cell is a human dendritic cell which may be immature or mature or at any other stage of development. Preferred types of dendritic cells include, but are not limited to, Langerhans cells. In one embodiment, the Langerhans cells contain Birbeck granules.

In some embodiments, the second agent is selected from the group consisting of proteins, polypeptides, peptides, macromolecules, chemical compounds, oligonucleotides and nucleic acids. In a preferred embodiment, the second agent is an antibody. The dendritic cell associated protein can be selected from the group of proteins consisting of CD1a, CD40, CD80, CD83, CD86, HLA-DR, HLA-A, HLA-B, HLA-C, CLA1, LFA-3, TLR-1, TLR-2, TLR-3, TLR-4 and TLR-5.

The dendritic cell associated protein in the extract can be on an array, or alternatively, the second agent is on an array comprising a plurality of agents capable of binding to at least one dendritic cell associated protein. In a preferred embodiment, the array is a biological chip.

The invention also encompasses methods of detecting the presence of a disease associated with altered dendritic cell activity in a patient comprising isolating a sample of dendritic cells from the patient; associating the sample with an agent which binds to a dendritic cell associated protein; measuring the amount of binding of the agent to the dendritic cell associated protein; wherein an alteration in the amount of binding is indicative of a disease associated with altered dendritic cell activity. In one embodiment, the amount of dendritic cell associated protein expressed is indicative of the extent of the disease.

In a preferred embodiment of this method, the dendritic cells are isolated from a patient suffering from a disease associated with altered dendritic cell activity. The disease can be an autoimmune disorder or due to an altered immune response. Preferred autoimmune disorders where the methods of the invention are applicable include, but are not limited to, psoriasis, inflammatory bowel disease, asthma, multiple schlerosis, lupus erythematosus, rheumatoid arthritis and type I diabetes. When the disease is due to an altered immune response, it can be a result of cancer or infectious disease.

The invention further encompasses a method of producing an antibody against a dendritic cell associated protein comprising isolating the dendritic cell associated protein; generating a phage display antibody library against the protein; associating the phage library with the dendritic cell associated protein; and isolating an antibody from the phage display antibody library that binds to the protein. In a preferred embodiment, the latter two steps are repeated to obtain an antibody which binds with higher affinity to the dendritic cell associated protein. The invention includes antibodies produced by the aforementioned methods of invention. In one embodiment, the antibody is monoclonal.

The invention also includes a method of screening for an agent that modulates the expression or activity of a dendritic cell associated protein comprising contacting a dendritic cell with a first agent which alters the expression or activity of a protein associated with the dendritic cell; contacting the dendritic cell with a second agent; and measuring the level of expression of the dendritic cell associated protein or mRNA encoding the dendritic cell associated protein; wherein an alteration in the level of expression indicates that the second agent modulates the expression or activity of the dendritic cell associated protein. In some embodiments, the level of expression is increased following contact with the second agent while in others it is decreased.

### BRIEF DESCRIPTION OF FIGURES

FIG. 1—Expression of cell-surface markers by human dendritic cell cultures

(A) CD34⁺ cells were seeded at 1×10⁴ cells/ml and cultured for eight days in the described growth medium (serum-free X-VIVO plus cytokines). Cells were then harvested, and the expression of HLA-DR, CLA, CD13, CD1a, CD14, and CD66b was determined by flow cytometry. Thin lines represent control antibodies. (B) Undisturbed control cells were stained at days ten (thin line) and twelve (thick line). Alternatively (right two panels) day ten cultures were harvested and recultured for two days at the same density in fresh growth medium (X-VIVO plus cytokines) or RPMI 1640 plus 5% fetal calf serum. Cells were stained for CD1a on day ten (thin line) and twelve (thick line). Dotted lines represent isotype controls. CD1a expression, consistent with the Langerhans cell phenotype was not lost upon reculture.

FIG. 2—Dendritic cells within cell clusters exhibit a Langerhans cell phenotype

Proliferating cultures (day eight) contain abundant multicellular aggregates of HLA-DR⁺ cells that express the Langerhans cell markers CD1a and Lag as shown by immunofluorescence microscopy (B & D), while single cells outside of the clusters generally expressed only HLA-DR (A & C). Note how one cell in the center of the cluster shown in (A) and (B), most likely a proliferating precursor, is negative for both differentiation markers. (E) Day ten cells were harvested and subjected to sedimentation on 7.5% bovine serum albumin columns resulting in an enrichment of CD1a⁺ cells from 55 to 89%. Cells were then replated for two days in the same medium. The majority (>90%) of cells after cluster purification and re-culture are also Lag⁺. (F) Electron microscopy analysis of Langerhans cell in culture reveals the presence of abundant Birbeck granules (indicated by arrowheads). (G) Only Bgs (and not other structures such as multilamellar lysosomes, indicated by asterisks) were heavily decorated by anti-Lag antibodies. However, the multilamellar structures did label with antibodies to MHC class II molecules (not shown).

FIG. 3—Maturation profile of human Langerhans cell-type dendritic cells

Purified clusters were replated at 5×10⁵ cell/well and cultured for two days. Replating was performed very carefully in order not to break the clusters (A) or after cluster disaggregation by repeated pipetting (B). Thin lines are cells stained immediately after cluster purification; thick lines are cells stained after two days in culture. Breakage of clusters induced spontaneous up-regulation of specific maturation markers. (C) Unbroken clusters can be induced to mature by addition of 250 ng/ml LPS (thin line) or 12.5 ng/ml TNF-alpha (thick line) to the regular growth medium (dotted line). (D) Clusters were cocultured with resting (thin line) or activated (thick line) platelets in RPMI-1640 supplemented with 5% fetal calf serum or simply replated in regular growth media (dotted line).

FIG. 4—Proinflammatory agents induce morphologically distinct Langerhans cells

Purified clusters were cultured for two days in fresh growth media alone (top row) or in presence of 12.5 ng/ml TNF-alpha (second row), 250 ng/ml LPS (third row) or activated platelets (bottom row). Cells were then fixed and stained for confocal immunofluorescence microscopy. In the merged images, HLA-DR staining is shown in green and Lamp-1 (CD107a) staining in red.

FIG. 5—Langerhans cell maturation induces enhanced CD4 and CD8 T cell alloreaction

Representative data from three experiments is shown. (A) Differentially treated dendritic cells were harvested and cocultured with freshly isolated CD8⁺ T cells. Proliferation at day two, four and six was measured by [³H]thymidine incorporation. (B) Proliferation of freshly isolated CD8⁺ and CD4⁺ T cells were measured in response to fixed dendritic cells. (C) IL-2 production was also measured for CD4⁺ T cells. (D) Proliferation at day six of CD4⁺ T cells (300,000 cells/well) was measured in response to the indicated amount of stimulating dendritic cell. (E) The ability of TNF-alpha and LPS to induce differential T cell responses did not reflect differences in the quantitative expression levels of surface markers. Surface staining for MHC class I, MHC class II, CD58 and CD83 are shown for control cells (dotted line), TNF-alpha treated cells (thick line) and LPS-treated cells (thin line).

FIG. 6—Differential expression of TLR genes after maturation by LPS & TNF-alpha

(A) Cluster-purified Langerhans cells were replated in X-VIVO (control, C) or X-VIVO containing LPS (L) or TNF-alpha (T) for forty-eight hours and expression of TLR1B5 was determined by RT-PCR. Amplification of actin mRNA was included as an internal control. (B) LPS-matured, but not TNF-matured, Langerhans cells produce IL-12. Cells were matured (as above) with either 12.5 ng/ml of TNF-alpha or 10 mg/ml of LPS. Supernatants were assayed for IL-12 content by ELISA. Representative data from two experiments is shown.

### DETAILED DESCRIPTION

Definitions

As used herein, the term “agent” refers to a peptide, polypeptide, protein, chemical compound, macromolecule and the like capable of binding to another molecule.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary.

As used herein, the term “Birbeck granule” refers to a small oblong-shaped, membrane bound granule characterized by cross-striated internal ultra-structure. Birbeck granules are normally found in Langerhans cells but not in any other cell type.

As used herein, the term “dendritic cell” refers to a professional antigen presenting cell, other than a B cell or macrophage, that initiates and controls the immune response.

As used herein, the term “dendritic cell associated protein” refers to any protein, polypeptide, peptide, macromolecule or the like, the expression of which by a particular type of dendritic cell is dependent upon contact of the dendritic cell with a particular agent either in vitro or in vivo. The expression can be either increased or decreased. Alternatively, in conditions where the expression is either increased or decreased in the absence of the agent, the presence of the agent can modulate the increase or decrease in expression such that no change in expression is observed.

As used herein, the term “Langerhans cell” refers to an epidermal dendritic cell.

As used herein, the term “protein” refers to any of a group of complex organic compounds which contain carbon, hydrogen, oxygen, nitrogen and usually sulphur, the characteristic element being nitrogen and which are widely distributed in plants and animals. Twenty different amino acids are commonly found in proteins and each protein has a unique, genetically defined amino acid sequence which determines its specific shape and function. The term “protein” as used herein, is synonomous with the terms peptide and polypeptide.

Methods for Producing Mature Dendritic Cells

In some embodiments, this invention encompasses a method of producing cultures of mature dendritic cells in vitro from immature dendritic cell precursors selected based on expression of the CD la antigen. In one embodiment, the method of the invention includes the generation of Langerhans cell subtypes by selective addition of particular agents during the maturation process. A unique embodiment of this invention is that Langerhans cell subtypes can be produced in large amounts suitable for screening purposes to identify cell surface molecules associated with a particular subset of these cells or particular disease states.

The starting material for the method of producing dendritic cell precursors and mature dendritic cells is a tissue source comprising CD34 antigen expressing dendritic cell precursors which are capable of proliferating and maturing in vitro into Langerhans cells when treated according to the methods of the invention. Such precursor cells are non-adherent and typically do not express antigens found on mature dendritic cells. Human blood is a preferred tissue source of precursor cells because it is easily accessible and can be obtained in relatively large quantities.

To increase the number of dendritic precursor cells in human blood, it is preferable to treat such individuals with substances which stimulate hematopoiesis. Such substances include G-CSF and GM-CSF as well as other factors which promote hematopoiesis. The amount of hematopoietic factor(s) to be administered may be determined by one skilled in the art by monitoring the cell differential of individuals to whom the factor is being administered. Typically, dosages of factors such as G-CSF and GM-CSF will be similar to the dosage used to treat individuals recovering from treatment with cytotoxic agents. Preferably, GM-CSF or G-CSF is administered daily at standard doses prior to removal of source tissue to increase the proportion of dendritic cell precursors.

In addition, the tissue source may be treated prior to culturing so as to enrich the proportion of dendritic precursor cells relative to other cell types. Such pretreatment may also remove cells which may compete with the proliferation of dendritic precursor cells or inhibit their proliferation or survival. Pretreatment may also be used to make the tissue source more suitable for in vitro culture. The method of treatment will likely be tissue specific depending on the particular tissue source. For example, treatment of blood would involve cell separation techniques to separate peripheral blood mononuclear cells from other cells types including red blood cells. Removal of red blood cells may be accomplished by standard methods known to those skilled in the art. In addition, methods such as apheresis can be used to isolate peripheral blood mononuclear cells from other blood constituents such as platelets and red blood cells.

In one form of pretreatment, a positive selection process is used where dendritic precursor cells are isolated and removed from other cells which compete and mask the proliferation of precursor dendritic cells. One such pretreatment comprises selecting for antigens specifically expressed on precursor cells and not expressed on other cells. In this way, cells can be immunoadsorbed onto magnetic beads coupled to an antibody which binds to a specific dendritic cell precursors and separated by magnetic force. Alternatively, the immunobeads can be separated by gravity. Non-adsorbed cells which are not dendritic cell precursors may then be separated from the cells adsorbed to the solid support by means standard in the art. These pretreatment steps serve a dual purpose because they remove precursors of non-dendritic cells in the culture while increasing the proportion of dendritic cell precursors.

In another form of the pretreatment, the antigen expressed by dendritic precursor cells is CD34. Dendritic precursor cells expressing this antigen can by positively selected and isolated from other cells using antibodies which bind CD34. The antibodies can be monoclonal or polyclonal. In one embodiment, fragments of antibodies which bind to CD34 can be used. The antibodies can be bound to any suitable matrix which facilitates the isolation of the dendritic cell precursors.

Another form of pretreatment to remove undesirable cells suitable for use with this invention is adsorbing the undesirable precursor cells or their precursors onto a solid support using antibodies specific for antigens expressed on the undesirable cells. Several methods of adsorbing cells to solid supports of various types are known to those skilled in the art and are suitable for use with this invention. The preferred embodiment of the present invention, however, is positive selection of cells by selecting for CD34 antigen expressing cells.

When blood is used as a tissue source, blood leukocytes may be obtained using conventional methods which maintain their viability. In a preferred embodiment, blood leukocytes are obtained by selecting for CD34 antigen expressing cells from leukapheresis products as previously described (Gatti et al., (2000) J. Immunol. 164, 3600-3607). CD34 antigen expressing cells can be immunomagnetically purified from leukapheresis products using any methods standard in the art. Preferred methods include, but are not limited to, the midi-MACS system (CD34 progenitor cell isolation kit or multisort kit) (Miltenyi Biotec) and the Isolex device (Baxter Scientific).

Alternatively, conventional methods can also be employed to isolate blood leukocytes. In one embodiment, blood is diluted into medium (preferably RPMI) containing heparin (about 100 units/ml) or other suitable anticoagulant. The volume of blood to medium is about one to one. Cells are pelleted and washed by centrifugation of the blood in medium at about 150×g at 4° C. Platelets and red blood cells are depleted by suspending the cell pellet in a mixture of medium and ammonium chloride. Preferably the mixture of medium to ammonium chloride is about one to one by volume. Cells are pelleted by centrifugation and washed about two more times in the medium-ammonium chloride mixture, or until a population of leukocytes, substantially free of platelets and red blood cells, is obtained.

Any isotonic solution commonly used in tissue culture may be used as the medium for separating blood leukocytes from platelets and red blood cells. Examples of such isotonic solutions are phosphate buffered saline, Hanks balanced salt solution, or complete growth mediums including for example X-VIVO. CD34⁺ cells obtained from treatment of the tissue source are cultured to form a primary culture on an appropriate substrate in a culture medium supplemented with one or more cytokines selected from the group consisting of GM-CSF, SCF, TNF-alpha, TGF-beta-1 and Flt3 ligand (Flt3L) or a derivative protein or peptide having an amino acid sequence which maintains the biologic activity typical of one or more of these cytokines. The appropriate substrate may be any tissue culture compatible surface to which cells may adhere. Preferably, the substrate is commercial plastic treated for use in tissue culture. Examples include various flasks, roller bottles, petri dishes and multi-well containing plates made for use in tissue culture. Surfaces treated with a substance, for example collagen or poly-L-lysine or antibodies specific for a particular cell type to promote cell adhesion may also be used provided they allow for the differential attachment of cells as described below. In one embodiment, cells are plated in twenty-four well plates at an initial cell density of about 2.5×10⁶ cells per well. At this dose, the surface is not fully covered by cells, but there are no big spaces (2-3 cell diameters) either.

The growth medium for the cells at each step of the method of the invention should allow for the survival and proliferation of the precursor dendritic cells. Any growth medium typically used to culture cells may be used according to the method of the invention provided the medium is supplemented with one or more of the cytokines selected from the group consisting of GM-CSF, SCF, TNF-alpha, TGF-beta-1 and Flt3 ligand. Preferred media includes X-VIVO, RPMI 1640 and DMEM, with added amino acids and vitamins supplemented with an appropriate amount of serum or a defined set of cytokines sufficient to promote proliferation of dendritic precursor cells. Serum-free medium supplemented with hormones is also suitable for culturing the dendritic cell precursors. X-VIVO supplemented with cytokines is preferred. Cells may be selected or adapted to grow in other serums and at other concentrations of serum. Cells from human tissue may also be cultured in medium supplemented with human serum rather than fetal calf serum. Medias may contain antibiotics to minimize bacteria infection of the cultures. Penicillin, streptomycin or gentamicin or combinations containing them are preferred. The medium, or a portion of the medium, in which the cells are cultured should be periodically replenished to provide fresh nutrients.

GM-CSF, SCF, TNF-alpha, TGF-beta-1 and Flt3 ligand have been found to promote the proliferation in vitro of precursor dendritic cells. Cells are cultured in the presence of one or more of these cytokines at a concentration sufficient to promote the survival and proliferation of dendritic cell precursors. The dose depends on the amount of competition from other cells (especially macrophages and granulocytes) for the cytokines, or to the presence of cytokine inactivators in the cell population. Preferably, the cells are cultured in the presence of between about 1 to 1000 units/ml of GM-CSF. More preferably cells from blood are cultured in the presence of GM-CSF at a concentration of between about 1.0 and 10 units/ml. This dose has been found to be necessary and sufficient for maximal responses by cells obtained from human blood. Most preferably, cells are cultured in the presence of GM-CSF at a concentration of about 5.6 units/ml.

When human blood leukocytes are cultured with these cytokines at the preferred concentrations, the cultures develop a large number of aggregates or cell balls from which typical dendritic cells are eventually released. In the absence of these cytokines, no colonies develop. Cytologic criteria may be used to initially detect the dendritic cells which characteristically extend large, sheet-like processes or veils (Strunk et al., (1997) J. Exp. Med. 185, 1131-1136; Inaba et al., (1992) J. Exp. Med. 176, 1693-1702; Henn et al., (1998) Nature 391, 591-594).

GM-CSF may be isolated from natural sources, produced using recombinant techniques or prepared by chemical synthesis. As used herein, GM-CSF includes GM-CSF produced by any method and from any species. GM-CSF is defined herein as any bioactive analog, fragment or derivative of the naturally occurring (native) GM-CSF. Such fragments or derivative forms of GM-CSF should also promote the proliferation in culture of dendritic cell precursors. In addition GM-CSF peptides having biologic activity can be identified by their ability to bind GM-CSF receptors on appropriate cell types.

It may be desirable to include additional agents in the culture medium in addition to GM-CSF to further increase the yield of dendritic cells. Such agents include G-CSF, M-CSF, IL-1-alpha, IL-1-beta, IL-3 and IL-6, TNF-alpha, Flt3 ligand and SCF. Such agents are used in amounts which are effective in increasing the proportion of dendritic cells present in the culture either by enhancing proliferation or survival of dendritic cell precursors. Preferably, the agents are present in the following concentrations: GM-CSF about 2.5×10⁻⁴ to 7.5×10⁻⁴ units/ml; SCF about 0.5 to 2.0 units/ml; TNF-alpha about 0.25 to 1.0 units/ml; Flt3 ligand about 50 to 150 ng/ml; and TGF-beta-1 about 0.05 to 0.2 units/ml. More preferred concentrations of agents are GM-CSF about 5×10⁻⁴ units/ml; SCF about 1.25 units/ml; TNF-alpha about 0.75 units/ml; Flt3 ligand about 100 ng/ml; and TGF-beta-1 about 1.25 units/ml. Preferred agents are human proteins. Preferred proteins are cytokines. Preferred cytokines are produced from the human gene using recombinant techniques.

The primary cultures from the tissue source are allowed to incubate at about 37° C. with 5% carbon dioxide under standard tissue culture conditions of humidity and pH until a population of cells has adhered to the substrate sufficiently to allow for the separation of non-adherent cells. Preferably, the primary cultures are incubated for about six to eleven days without feeding or replating or until a sufficient time such that the total cell number has increased about fifty-fold, preferably about one hundred-fold. Non-adherent cells appear as clusters which can be isolated by gently harvesting cells with a pipette and layering the harvested cells on top of a media comprising about five to ten percent bovine serum albumin, preferably about seven to eight percent bovine serum albumin. The cell cluster suspension is preferably incubated on ice for a sufficient time followed by removal of single cells by aspiration. Cell clusters can then be concentrated by centrifugation, resuspended in growth media and cultured for about an additional two days.

To culture precursor cells from human blood from this primary culture, cells which have been depleted of cells that are not dendritic cell precursors are cultured on a substrate at a density of preferably about 5×10⁵ cells/ml/well in twenty-four well plates. After five days, with feedings every other day, cell aggregates appear. These aggregates may then be treated as described below.

The non-adherent cells from the primary culture are subcultured by transferring them to new culture flasks at a density sufficient to allow for survival of the cells and which results in the development over time of clusters of growing cells that are loosely attached to the culture surface or to the firmly adherent cells on the surface. These clusters are the nidus of proliferating dendritic cell precursors. As used herein “culture flask” refers to any vessel suitable for culturing cells. It is desirable to subculture all of the non-adherent cells from the primary culture at a density of between about 2×10⁵ cells and 5×10⁵ cells per cm². Preferably at about 2.5×10⁵ cells per cm². Cells are incubated for a sufficient time to allow the surface of the culture dish to become covered with a monolayer of tightly adherent cells including macrophages and fibroblasts affixed to which are aggregates of non-adherent cells. At this time, any non-adherent cells are removed from the wells, and the cellular aggregates are dislodged for subculturing. Preferably the cells from the aggregates are subcultured after about ten days or when the number of aggregated cells per cm² reaches about 3×10⁵.

For serially subculturing the aggregated cells, the aggregated cells are dislodged from the adherent cells and the aggregated cells are subcultured on a total surface area of preferably between about two to five times that of the surface area of the parent culture. More preferably the cells are subcultured on a surface area that is about three times the surface area of the parent culture. Cells having sheet-like processes typical of dendritic cells appear in the culture at about four to seven days. Between about day ten and seventeen of culture the number of single cells that can be recovered from a given surface area doubles. Both dendritic cell precursors and mature dendritic cells are present in the aggregates.

To further expand the blood derived population of dendritic cells, cell aggregates may be serially subcultured multiple times at intervals which provide for the continued proliferation of dendritic cell precursors. Preferably, aggregates are subcultured prior to the release into the medium of a majority of cells having the dendritic cell morphology, for example between about three and thirty days. More preferably aggregates of cells are subcultured between about ten to twenty-five days in culture, and most preferably at twenty days. The number of times the cells are serially subcultured depends on the number of cells desired, the viability of the cells, and the capacity of the cultures to continue to produce cell aggregates from which dendritic cells are released. Preferably, cells can be serially subcultured for between about one to two months from when the nonadherent cells were subcultured or between about one to five times. More preferably cells are serially subcultured about two to three times. Most preferably cells are serially subcultured twice.

According to a preferred method, to serially subculture the cells of the primary and subsequent cultures, cells are dislodged by pipetting most of the aggregates of growing dendritic cells as well as some cells in the monolayer of growing macrophages and fibroblasts. Pipetting usually disrupts the aggregates, particularly the peripheral cells of the aggregates which are more mature. With time in culture (e.g., two weeks), the aggregates of the growing dendritic cells become more stable and it is possible to dislodge the aggregates for separation by 1×g sedimentation.

Alternative approaches may be used to isolate the mature dendritic cells from the growing cultures. One approach is to remove cells that are non-adherent and separate the aggregates from cells attached to substrate and single cells by 1×g sedimentation. Dendritic cells are then released in large numbers from the aggregates over an additional one to two days of culture, while any mature dendritic cells can be isolated from other single cells by floatation on dense metrizamide as described (Freudenthal & Steinman, (1990) Proc. Natl. Acad. Sci. USA 87, 7698-7702). The second method, which is simpler but essentially terminates the growth phase of the procedure, is to harvest all the non-adherent cells when the aggregates are very large, leave the cells on ice for about twenty minutes, resuspend vigorously with a pipette to disaggregate the aggregates and float the mature dendritic cells on metrizamide columns.

Various techniques may be used to identify the cells present in the cultures. These techniques may include analysis of morphology, detecting cell type specific antigens with monoclonal antibodies, identifying proliferating cells using tritiated thymidine autoradiography, assaying mixed leukocyte reactions and demonstrating dendritic cell homing.

The dendritic cells besides being identified by their stellate shape, may also be identified by detecting their expression of specific antigens using monoclonal antibodies. A panel of monoclonal antibodies may be used to identify and characterize the cells in the cytokine expanded cultures. Among the specific monoclonal antibodies suitable for identifying mature dendritic cells are those which bind to the MHC class I & II antigens, those which bind to heat stable antigen, anti-dendritic cell antibodies and those which bind to antigens in granules in the perinuclear region of mature dendritic cells (Agger et al., (1990) Int. Rev. Immunol. 6, 89-101). Other antigens which are expressed by dendritic cells and which may be used to identify mature dendritic cells are CD44 and CD11b. Those of skill in the art will recognize that other antibodies may be made and characterized which are suitable for identifying mature dendritic cells. Similarly, the production of dendritic precursor cells also facilitates the production of antibodies specific for dendritic precursor cells.

To identify and phenotype the proliferating cells and their progeny, cultures may be labeled with tritiated thymidine to identify the cells in the S phase of mitosis. In addition to labeling the cells with a mitotic label, cells may also be co-labeled with monoclonal antibodies to determine when markers associated with mature dendritic cells are expressed. The distinctive phenotype of the dendritic cell precursors is stable so that for example, the dendritic cell progeny do not become macrophages even when maintained in macrophage colony stimulating factor (M-CSF).

Another index of dendritic cell maturity is the ability of mature dendritic cells to stimulate the proliferation of T-cells in the mixed leukocyte reaction (MLR). The ability of dendritic cells to migrate to lymph nodes (i.e., dendritic cell homing is another index of dendritic cell maturation which may be used to assess the maturity of the cells in culture). The criteria that have become evident for identifying dendritic precursor cells according to the invention enable the identification of proliferating progenitors of dendritic cells in other organs. The proliferation of leukocytes occurs in the bone marrow, but it may be that for dendritic cells, the marrow also seeds the blood and other tissues with progenitors which then proliferate extensively as shown here. By being able to prepare the otherwise trace dendritic cell in large numbers and scale according to the method of this invention, other previously unexplored areas of dendritic cell function may now be determined. Specifically, growing dendritic cells will facilitate molecular and clinical studies on the expression of cell surface molecules which are specific for a particular dendritic cell subtype associated with a particular tissue or disease state.

Antigen-activated dendritic cells of the invention are produced by exposing antigen, in vitro, to the dendritic cells prepared according to the method of the invention. Dendritic cells are plated in culture dishes and exposed to antigen in a sufficient amount and for a sufficient period of time to allow the antigen to bind to the dendritic cells. The amount and time necessary to achieve binding of the antigen to the dendritic cells may be determined by immunoassay or binding assay. Other methods known to those of skill in the art may be used to detect the presence of antigen on the dendritic cells following their exposure to antigen.

Processing of antigen by dendritic cells or dendritic cell precursors includes the fragmentation of an antigen into antigen fragments which are then presented. Phagocytoses of particulate matter by dendritic cell precursors may be accomplished by culturing the dendritic cell precursors in the presence of particulate matter for a time sufficient to allow the cells to phagocytose, process and present the antigen. Preferably, culturing of the cells in the presence of the particles should be for a period of between one to forty-eight hours. More preferably, culturing cells in the presence of particulate matter will be for about twenty hours. Those of skill in the art will recognize that the length of time necessary for a cell to phagocytose a particle will be dependent on the cell type and the nature of the particle being phagocytosed. Methods to monitor the extent of such phagocytosis are well known to those skilled in the art.

Cells should be exposed to antigen for sufficient time to allow antigens to be internalized and presented on the cell surface. The time necessary for the cells to internalize and present the processed antigen may be determined using pulse-chase protocols in which exposure to antigen is followed by a wash-out period. Once the minimum time necessary for cells to express processed antigen on their surface is determined, a pulse-chase protocol may be used to prepare cells and antigens for eliciting immunogenic responses. The phagocytic dendritic precursor cells are obtained by stimulating cell cultures comprising dendritic precursor cells with cytokines to induce aggregates of growing dendritic cells. These dendritic precursor cells may be obtained from any of the source tissues containing dendritic cell precursors described above. Preferably, the source tissue is blood. Cells within these aggregates are clearly phagocytic.

In effect, the pulse and chase protocol which may be used to charge developing dendritic cells with organisms according to our invention allows the two broad components of immunostimulation to take place sequentially. These components are antigen capture and presentation, here the capture of particulates by immature dendritic cells, and development of potent accessory or immunostimulatory functions during the chase period. Examples of such protocols are well known and include the processing of soluble proteins (Romani et al., (1989) J. Exp. Med. 169, 1169-1178; Pure et al., (1990) J. Exp. Med. 172, 1459-1469) by epidermal Langerhans cells. Each of the two broad components of antigen processing cell function entails many subcomponents.

Therapeutic Methods Using Dendritic Cells

According to this embodiment of the invention, the proliferating dendritic cells may be injected with a vector which allows for the expression of specific antigenic proteins by the dendritic cells. These antigenic proteins which are expressed by the dendritic cell may then be processed and presented on the cell surface on MHC I receptors. The antigen-presenting cells or the processed antigens themselves may then be used as immunogens to produce an immunogenic response to the proteins encoded by the vector.

Vectors can be prepared to include specific nucleic acid sequences which code and express genes for proteins to which an immunogenic response is desired. Preferably, retroviral vectors are used to infect the dendritic cells. The use of retroviral vectors to infect host cells is known to those skilled in the art.

By using developing dendritic cells to activate MHC class I and II products, several desirable components of T cell modulation in situ can be achieved. Antigen uptake and presentation by immature progenitors, allows the antigen processing cell to tailor the peptides that are appropriate for an individual's MHC products and increases the number of specialized stimulatory antigen processing cells. These properties of dendritic cell progenitor populations meet many of the demands for using cells as vehicles for active immunization and immunotherapy in situ. The present invention provides a method of obtaining dendritic cells in sufficient quantities to be used to treat or immunize animals or humans with dendritic cells which have been activated with antigens. In addition, dendritic cells may be obtained in sufficient quantities to be useful as reagents to modify antigens in a manner to make the antigens more effective as T-cell dependent antigens.

To use antigen-activated dendritic cells as a therapeutic or immunogen the antigen-activated dendritic cells are injected into a human by any method which elicits an immune response. Preferably, dendritic cells are injected back into the same human from whom the source tissue was obtained. The injection site may be subcutaneous, intraperitoneal, intramuscular, intradermal or intravenous. The number of antigen-activated dendritic cells reinjected back into the human in need of treatment may vary depending on the antigen and size of the individual. A key feature in the function of dendritic cells in situ is the capacity to migrate or home to the T cell-dependent regions of lymphoid tissues, where the dendritic cells would be in an optimal position to select the requisite antigen-reactive T cells from the pool of recirculating quiescent lymphocytes and thereby initiate the T cell-dependent response.

According to the preferred method of stimulating an immune response in an individual, a tissue source from that individual would be identified to provide the dendritic cell precursors. If blood is used as the tissue source preferably the individual is first treated with cytokine to stimulate hematopoieses. After isolation and expansion of the dendritic cell precursor population, the cells are contacted with a preselected antigen. Preferably, contact with the antigen is conducted in vitro. After sufficient time has elapsed to allow the cells to process and present the antigen on their surfaces, the cell-antigen complexes are put back into the individual in sufficient quantity to evoke an immune response. Preferably between 1×10⁶ and 10×10⁶ antigen presenting cells are injected back into the individual.

The preselected antigens are prepared by combining substances to be modified or other antigens with the dendritic cells prepared according to the method of the invention. The dendritic cells process or modify antigens in a manner which promotes the stimulation of T cells by the processed or modified antigens. Such dendritic cell modified antigens are advantageous because they can be more specific and have fewer undesirable epitopes than non-modified T cell-dependent antigens. The dendritic cell modified antigens may be purified by standard biochemical methods. For example, it is known to use antibodies to products of the MHC to select MHC-antigenic peptide complexes and then to elute the requisite processed peptides with acid (Rudensky et al., (1991) Nature 353, 622-627; Hunt et al., (1992) Science 255, 1261-1263 which are incorporated herein by reference in their entirety).

Antigen-activated dendritic cells and dendritic cell modified antigens may both be used to elicit an immune response against an antigen. The activated dendritic cells or modified antigens may by used as vaccines to prevent future infection or may be used to activate the immune system to treat ongoing disease. The activated dendritic cells or modified antigens may be formulated for use as vaccines or pharmaceutical compositions with suitable carriers such as physiological saline or other injectable liquids. The vaccines or pharmaceutical compositions comprising the modified antigens or the antigen-activated dendritic cells of the invention would be administered in therapeutically effective amounts sufficient to elicit an immune response. Preferably, between about 1 to 100 micrograms of modified antigen, or its equivalent when bound to dendritic cells, should be administered per dose.

Because dendritic cells can now be grown from precursors according to the methods identified here, and because dendritic cells can modify antigens to produce killer T cells, the compositions of this invention are particularly useful as vaccines towards viruses and tumor cells for which killer T cells might provide resistance.

Identification of a Dendritic Cell Associated Protein

In one embodiment, a dendritic cell associated protein is identified using a phage display method (McCafferty et al., (1990) Nature 348, 552-554). In such a method, display of recombinant proteins having heavy chain framework sequences and random hypervariable regions on the surface of viruses which infect bacteria (bacteriophage or phage) make it possible to produce soluble, recombinant proteins having human hypervariable regions with a wide range of affinities and kinetic characteristics. To display the recombinant proteins on the surface of phage (phage display), a hypervariable region gene is inserted into the gene encoding a phage surface protein (pIII) and the recombinant fusion protein is expressed on the phage surface (McCafferty et al., (1990) Nature 348, 552-554; Hoogenboom et al., (1991) Nucleic Acids Res. 19, 4133-4137).

Since the recombinant proteins on the surface of the phage are functional, phage bearing antigen binding hypervariable regions can be separated from non-binding or lower affinity phage by antigen affinity chromatography. Mixtures of phage are allowed to bind to the affinity matrix, non-binding or lower affinity phage are removed by washing, and bound phage are eluted by treatment with acid or alkali. Depending on the affinity of the hypervariable region of the recombinant protein, enrichment factors of twenty-fold to a million-fold are obtained by single round of affinity selection. By infecting bacteria with the eluted phage, however, more phage can be grown and subjected to another round of selection. In this way, an enrichment of a thousand-fold in one round becomes a million-fold in two rounds of selection. Thus, even when enrichments in each round are low (Marks et al., (1991) J. Mol. Biol, 222, 581-597), multiple rounds of affinity selection leads to the isolation of rare phage and the genetic material contained within which encodes the sequence of the hypervariable region of the recombinant binding protein.

In one embodiment of the invention, methods are disclosed for producing a phage expression library encoding antibodies having heavy chain framework sequences and random hypervariable regions capable of binding to a protein or proteins that are selectively expressed by a dendritic cell. In a further embodiment, phage display can be used to identify proteins selectively expressed by dendritic cells, preferably a subset of dendritic cells, even more preferably, Langerhans cells, most preferably, a subset or subtype of Langerhans cells. In another embodiment, the antibodies can be used to identify and isolate a dendritic cell subtype or subset which expresses a protein epitope to which the hypervariable region of the recombinant protein binds.

Structural modifications that include the addition of restriction enzyme recognition sites into the polynucleotide sequence encoding the antibodies which enable genetic manipulation of the hypervariable gene sequences are also encompassed in the invention. The re-engineered heavy chain gene sequences can be ligated into an M13-derived bacteriophage cloning vector that permits expression of a fusion protein on the phage surface. These methods allow for selecting phage clones encoding fusion proteins that bind a target ligand and can be completed in a rapid manner allowing for high-thoroughput screening of dendritic cell preparations.

According to the methods of the invention, a library of phage displaying modified heavy chain proteins is incubated with the immobilized proteins to select clones encoding recombinant proteins that specifically bind the immobilized protein. This procedure involves immobilizing a protein or polypeptide sample from a dendritic cell on a solid substrate. The bound phage are then dissociated from the immobilized ligand and amplified by growth in bacterial host cells. Individual viral plaques, each expressing a different recombinant protein, are expanded to produce amounts of protein sufficient to perform an inhibition assay. The DNA encoding this recombinant binding protein can be subsequently modified for ligation into a eukaryotic protein expression vector. The mutant heavy chain gene, adapted for expression in eukaryotic cells, is ligated into a eukaryotic protein expression vector.

Phage display methods that can be used to make the antibodies of the present invention include those disclosed in Brinkman et al., (1995) J. Immunol. Methods 182, 41-50; Ames et al., (1995) J. Immunol. Methods 184:177-186; Kettleborough et al., (1994) Eur. J. Immunol. 24, 952-958; Persic et al., (1997) Gene 187, 9-18; Burton et al., (1994) Adv. Immunol. 57, 191-280; U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743, 5,837,500 & 5,969,108 each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, yeast and bacteria. For example, techniques to recombinantly produce Fab, Fab′ and F(ab′)₂ fragments can also be employed using methods in the art such as those disclosed in Better et al., (1992) WO 92/22324 and Mullinax et al., (1992) Biotechniques 12, 864-869 these references incorporated by reference in their entirety).

Those having ordinary skill in the art will appreciate that the proteins selectively expressed by dendritic cells which bind by the recombinant binding proteins can be carbohydrates, lipids, proteins or nucleic acids. Further, these ligands can be either intracellular constituents or extracellular molecules. Intracellular constituents are represented by molecular species located within a cell. Such intracellular constituents can, for example, be enzymes, components of transmembrane signaling complexes, or transcription factors. Extracellular targets contemplated to fall within the scope of the invention include molecules that are present on the cell surface or are secreted by a cell. Such molecules may, for example, be cell lineage-specific proteins, glycoproteins and transmembrane proteins.

Isolation of Dendritic Cell Associated Proteins

In one embodiment, the invention provides a method for identifying and isolating a dendritic cell associated protein following induction with a particular agent, preferably a subset of dendritic cell, even more preferably Langerhans cell and most preferably a subset of Langerhans cells.

Once a dendritic cell associated protein is identified and isolated, the nucleic acid which encodes the protein, a portion of the protein or a precursor of the protein is identified, the gene product can be analyzed. This is achieved by assays based on the physical or functional properties of the product, including radioactive labeling of the product followed by analysis by gel electrophoresis, immunoassay, etc.

The dendritic cell associated protein identified herein can be isolated and purified by standard methods including chromatography (e.g., ion exchange, affinity and sizing column chromatography), centrifugation, differential solubility or by any other standard technique for the purification of proteins.

Alternatively, the nucleic acid that encodes the dendritic cell associated protein can first be identified, then the entire amino acid sequence of the protein can be deduced from the nucleotide sequence of the gene coding region contained in the nucleic acid. As a result, the protein can be synthesized by standard chemical methods known in the art (e.g., see Hunkapiller et al., (1984) Nature 310, 105-111).

In another alternative embodiment, a dendritic cell associated protein can be purified from natural sources by standard methods such as those described above (e.g., immunoaffinity purification). In one embodiment, the proteins are isolated by preparative-scale runs, a narrow-range “zoom gel” having a pH range of two units or less is preferred for the isoelectric step, according to the method described in Westermeier, (1993) Electrophoresis in Practice, VCH Publishing, herein incorporated herein by reference in its entirety. This modification permits a larger quantity of a target protein to be loaded onto the gel, and thereby increases the quantity of isolated proteins that can be recovered from the gel. Those of skill in the art will appreciate that a zoom gel can be used in any separation strategy which employs gel isoelectric focusing.

Identification of Nucleic Acids Encoding a Dendritic Cell Associated Protein

The invention encompasses methods for identification of nucleic acids encoding a dendritic cell associated protein, preferably from a subset of dendritic cells, more preferably, Langerhans cells, even more preferably, a subset or subtype of Langerhans cell. The present invention allows for identification of these proteins by providing methods for isolation of nucleic acid molecules that encode proteins or families of dendritic cell associated proteins which are expressed by human Langerhans cells or subsets of human Langerhans cells.

Essentially, a skilled artisan can readily use the amino acid sequence of a dendritic cell associated protein to generate antibody probes to screen expression libraries prepared from appropriate cells. In a preferred embodiment, the appropriate cells are human dendritic cells, more preferably, Langerhans cells. Typically, polyclonal antiserum from mammals such as rabbits immunized with the purified protein or monoclonal antibodies can be used to probe a mammalian cDNA or genomic expression library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence can be synthesized and used as a probe to retrieve DNA encoding a member of the protein family from various tissue sources including diseased tissue. Oligomers containing approximately 18-20 nucleotides (encoding about a six to seven amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Antibodies Against a Dendritic Cell Associated Protein

According to the invention, an isolated dendritic cell associated protein or a fragment or derivative thereof may be used as an immunogen to generate antibodies which immunospecifically bind the protein. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules (i.e., molecules that contain an antigen binding site that specifically binds an antigen). The immunoglobulin molecules of the invention can be of any class (e.g., IgG, IgE, IgM, IgD and IgA) or subclass of immunoglobulin molecule. In one embodiment of the invention, antibodies to a specific domain of a protein selectively expressed by a dendritic cell are produced.

In the production of antibodies, screening for the desired antibody can be accomplished by techniques known in the art (e.g., ELISA). For example, to select antibodies which recognize a specific domain of a dendritic cell associated protein, one may assay generated hybridomas for a product which binds to a fragment containing such a domain. For selection of an antibody that specifically binds a first dendritic cell associated protein, but which does not specifically bind to (or binds less avidly to) a second dendritic cell associated protein, one can select on the basis of positive binding to the first protein and lack of binding to (or reduced binding to) the second protein. Similarly, for selection of an antibody that specifically binds a dendritic cell associated protein but which does not specifically bind to (or binds less avidly to) a different isoform of the same protein (such as a different glycoform), one can select on the basis of positive binding to the protein and a lack of binding to (or reduced binding to) the different isoform (e.g., a different glycoform). Thus, the present invention provides an antibody that binds with greater affinity (preferably at least two-fold, more preferably at least five-fold, still more preferably at least ten-fold greater affinity) to such a protein than to a different isoform or isoforms (e.g., glycoforms) of the protein.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a dendritic cell associated protein, a fragment thereof, a related polypeptide, or a fragment of a related polypeptide. In a particular embodiment, rabbit polyclonal antibodies to an epitope of a related polypeptide can be obtained. For example, for the production of polyclonal or monoclonal antibodies, various host animals can be immunized by injection with the native or a synthetic (e.g., recombinant) version of a protein selectively expressed by a dendritic cell, a fragment thereof, a related polypeptide or a fragment of a related polypeptide, including but not limited to rabbits, mice, rats, etc. If the protein is purified by gel electrophoresis, the protein can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol and an adjuvant such as BCG (bacille Calmette-Guerin) or Corynebacterium parvum. Additional adjuvants are also well known in the art.

For preparation of monoclonal antibodies directed toward a dendritic cell associated protein, a fragment thereof, a related polypeptide or a fragment of a related polypeptide, any technique which provides for the production of antibody molecules by continuous cell lines in culture may be used. For example, the hybridoma technique originally developed by Kohler & Milstein, (1975) Nature 256, 495-497), as well as the trioma technique, the human B cell hybridoma technique (Kozbor et al., (1983) Eur. J. Immunol. 14, 23-27) and the EBV-hybridoma technique to produce human monoclonal antibodies (Cole et al., (1985) in Monoclonal Antibodies and Cancer Therapy, Liss Publishers). Such antibodies may be of any immunoglobulin class including IgG, IgM, IgE, IgA, IgD and any subclass thereof. The hybridoma producing the monoclonal antibodies of the invention may be cultivated in vitro or in vivo.

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine monoclonal antibody (e.g., U.S. Pat. Nos. 4,816,567 & 4,816,397 which are incorporated herein by reference in their entirety). Humanized antibodies are antibody molecules from non-human species having one or more complementarily determining regions from the non-human species and a framework region from a human immunoglobulin molecule (e.g., U.S. Pat. No. 5,585,089 which is incorporated herein by reference in its entirety.)

Chimeric and humanized monoclonal antibodies can be produced by recombinant DNA techniques known in the art, for example using methods described in Liu et al., (1987) WO 87/02671; Neuberger et al., (1986) WO 86/01533; U.S. Pat. No. 4,816,567; Better et al., (1988) Science 240, 1041-1043; Liu et al., (1987) Proc. Natl. Acad. Sci. USA 84, 3439-3443; Liu et al., (1987) J. Immunol. 139, 3521-3526; Sun et al., (1987) Proc. Natl. Acad. Sci. USA 84, 214-218; Nishimura et al., (1987) Canc. Res. 47, 999-1005; Wood et al., (1985) Nature 314, 446-449.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen. Monoclonal antibodies directed against the antigen can be obtained using conventional hybridoma technology. The human immunoglobulin transgenes harbored by the transgenic mice rearrange during B cell differentiation and subsequently undergo class switching and somatic mutation. Thus, using such a technique, it is possible to produce therapeutically useful IgG, IgA, IgM and IgE antibodies. For an overview of this technology for producing human antibodies, see Lonberg & Huszar, (1995) Int. Rev. Immunol. 13, 65-93. For a detailed discussion of this technology for producing human antibodies and human monoclonal antibodies and protocols for producing such antibodies, see, e.g., U.S. Pat. Nos. 5,625,126; 5,633,425; 5,569,825; 5,661,016 & 5,545,806.

Completely human antibodies which recognize a selected epitope can be generated using a technique referred to as a “guided selection” approach. In this approach a selected non-human monoclonal antibody, e.g., a mouse antibody, is used to guide the selection of a completely human antibody recognizing the same epitope (Jespers et al., (1994) Biotechnology 12, 899-903).

The antibodies of the present invention can also be generated using various phage display methods known in the art as described elsewhere in the specification. In phage display methods, functional antibody domains are displayed on the surface of phage particles which carry the polynucleotide sequences encoding them. In particular, such phage can be utilized to display antigen binding domains expressed from a repertoire or combinatorial antibody library (e.g., human or murine). Phage expressing an antigen binding domain that binds the antigen of interest can be selected or identified with antigen (e.g., using labelled antigen or antigen bound or captured to a solid surface or bead). Phage used in these methods are typically filamentous phage including fd and M13 binding domains expressed from phage with Fab, Fv or disulfide stabilized Fv antibody domains recombinantly fused to either the phage gene III or gene VIII protein.

The invention further provides for the use of bispecific antibodies, which can be made by methods known in the art. Traditional production of full length bispecific antibodies is based on the co-expression of two immunoglobulin heavy chain-light chain pairs, where the two chains have different specificities (Milstein et al., (1983) Nature 305, 537-539). Because of the random assortment of immunoglobulin heavy and light chains, these hybridomas (quadromas) produce a potential mixture of ten different antibody molecules, of which only one has the correct bispecific structure. Similar procedures are disclosed in Berg et al., (1993) WO 93/08829 and in Traunecker et al., (1991) EMBO J. 10, 3655-3659.

In another approach encompassed by the invention, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. Nucleic acids encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in Ashkenazi et al., (1994) WO 94/04690 incorporated herein by reference in its entirety.

The invention provides functionally active fragments, derivatives or analogs of the antibodies of the invention. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and complementary determining region sequences that are C-terminal to the complementary determining region sequence that specifically recognizes the antigen. To determine which complementary determining region sequences bind the antigen, synthetic peptides containing the complementary determining region sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fv or single chain antibodies (e.g., U.S. Pat. No. 4,946,778; Bird, (1988) Science 242, 423-426; Huston et al., (1988) Proc. Natl. Acad. Sci. USA 85, 5879-5883; Ward et al., (1989) Nature 334, 544-554) or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., (1988) Science 242, 1038-1041).

In other embodiments, the invention provides fusion proteins of the antibody of the invention (or functionally active fragments thereof), for example in which the antibody is fused via a covalent bond (e.g., a peptide bond), at either the N-terminus or the C-terminus to an amino acid sequence of another protein (or portion thereof, preferably at least a ten, twenty or fifty amino acid portion of the protein) that is not the antibody. In one embodiment, the antibody or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The antibodies of the invention include analogs and derivatives that are either modified i.e., by the covalent attachment of any type of molecule, as long as such covalent attachment that does not impair immunospecific binding. For example, but not by way of limitation, the derivatives and analogs of the antibodies include those that have been further modified (e.g., by glycosylation, acetylation, pegylation, phosphylation, amidation, derivatization by known protecting and blocking groups, proteolytic cleavage, linkage to a cellular ligand or other protein). Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of a dendritic cell associated protein (e.g., for imaging these proteins, measuring levels thereof in appropriate physiological samples, in diagnostic methods).

The antibody produced by the methods of the invention can also be used to validate activation of a dendritic cell by a particular cytokine. For example, following incubation of immature dendritic cells with a selected cytokine, the antibody can be used to confirm that activation occurred by contacting the antibody with the activated cells and confirming expression of a selected dendritic cell associated protein which is only expressed following activation.

Finally, the antibody can be humanized to identify and isolate in vivo, altered activity of dendritic cells associated with a particular disease by selectively binding to such cells following administration to an individual. The antibody can also be used as a diagnostic and therapeutic agent to target and destroy these cells with altered activity. Such disease states include, but are not limited to, autoimmune disorders or diseases assoiciated with an altered immune response. Autoimmune disorders where the antibody will be useful include, but are not limited to, psoriasis, inflammatory bowel disease, asthma, multiple schlerosis, lupus erythematosus, rheumatoid arthritis and type I diabetes. Diseases associated with an altered immune response where the antibody is useful include, but are not limited to cancer and infectious disease.

Recombinant Antibody Production

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., (1994) Biotechniques 17, 242-246) which briefly involves the synthesis of overlapping oligonucleotides containing portions of the sequence encoding antibody, annealing and ligation of those oligonucleotides and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridized to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., (1989) Science 246, 1275-1281) for clones of Fab fragments that bind the specific antigen or by screening antibody libraries (e.g., Clackson et al., (1991) Nature 352, 624-628; Hane et al., (1997) Proc. Natl. Acad. Sci. USA 94, 4937-4942).

Once a nucleic acid encoding at least the variable domain of the antibody molecule is obtained, it may be introduced into a vector containing the nucleotide sequence encoding the constant region of the antibody molecule (e.g., Boss et al., (1986) WO 86/05807; Bebbington et al., (1989) WO 89/01036; U.S. Pat. No. 5,122,464). Vectors containing the complete light or heavy chain for co-expression with the nucleic acid to allow the expression of a complete antibody molecule are also available. Then the nucleic acid encoding the antibody can be used to introduce the nucleotide substitutions or deletions necessary to substitute (or delete) the one or more variable region cysteine residues participating in an intra-chain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., (1978) J. Biol. Chem. 253, 6551-6560), PCR based methods, etc.

In addition, techniques developed for the production of “chimeric antibodies” (Morrison et al., (1984) Proc. Natl. Acad. Sci. USA 81, 851-855; Neuberger et al., (1984) Nature 312, 604-608; Takeda et al., (1985) Nature 314, 452-454) by splicing genes from a mouse antibody molecule of appropriate antigen specificity together with genes from a human antibody molecule of appropriate biological activity can be used. As described herein, a chimeric antibody is a molecule in which different portions are derived from different animal species, such as those having a variable region derived from a murine mAb and a human antibody constant region (e.g., humanized antibodies).

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination (e.g., Sambrook et al., (1990) Molecular Cloning—A Laboratory Manual, Cold Spring Harbor Laboratory Press; Ausubel et al., (1998) Current Protocols in Molecular Biology, Wiley). The expression vector can be transferred to a host cell by conventional techniques and the transfected cells can then cultured by conventional techniques to produce an antibody of the invention.

The host cells used to express a recombinant antibody of the invention may be either bacterial cells such as E. coli, or alternatively, eukaryotic cells, especially for the expression of whole recombinant antibody molecule. In particular, mammalian cells such as Chinese hamster ovary cells (CHO) in conjunction with a vector such as the major intermediate early gene promoter element from human cytomegalovirus is an effective expression system for antibodies (Foecking et al., (1986) Gene 45, 101-105).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter).

In bacterial systems, a number of expression vectors may be advantageously selected depending upon the use intended for the antibody molecule being expressed. For example, when a large quantity of such a protein is to be produced for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., (1983) EMBO J. 2, 1791-1794) in which the antibody coding sequence may be ligated individually into the vector in frame with the lacZ coding region so that a fusion protein is produced; pIN vectors (Inouye & Inouye, (1985) Nucleic Acids Res. 13, 3101-3109; Van Heeke & Schuster, (1989) J. Biol. Chem. 24, 5503-5509) and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin) and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington & Hentschel, (1987) The use of vectors based on gene amplification for the expression of cloned genes in mammalian cells in DNA cloning, Academic Press). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., (1983) Mol. Cell. Biol. 3, 257-266).

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Kohler, (1980) Proc. Natl. Acad. Sci. USA 77, 2197-2179). The coding sequences for the heavy and light chains may comprise cDNA or genomic DNA.

Once the antibody of the invention has been recombinantly expressed, it may be purified by any method known in the art for purification of an antibody molecule, for example, by chromatography (e.g., ion exchange chromatography, affinity chromatography such as with protein A or specific antigen and sizing column chromatography), centrifugation, differential solubility or by any other standard technique for the purification of proteins.

Alternatively, any fusion protein may be readily purified by utilizing an antibody specific for the fusion protein being expressed. For example, systems described have been described which allow for the ready purification of non-denatured fusion proteins expressed in human cell lines (Janknecht et al., (1991) Proc. Natl. Acad. Sci. USA 88, 8972-8976). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Nickel nitriloacetic acid-agarose columns and histidine-tagged proteins are selectively eluted with imidazole-containing buffers.

Methods to Identify Binding Partners

Another embodiment of the present invention provides methods for use in isolating and identifying binding partners of a dendritic cell associated protein. In detail, a potential binding partner is contacted with an extract or fraction of a cell under conditions that allow the association of potential binding partners with the dendritic cell associated protein. After mixing, peptides, polypeptides, proteins or other molecules that have become associated with the potential binding partner are separated from the mixture. The binding partner that bound to the protein can then be removed and further analyzed.

As used herein, a “cellular extract” refers to a preparation or fraction which is made from a lysed or disrupted cell. The preferred source of cellular extracts will be derived from human dendritic cells, even more preferably, human Langerhans cells. In some embodiments, the extract is a cell membrane extract while in others it is a cytosolic or nuclear extract.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with an agent presumed to be a potential binding partner under conditions in which association of the protein with the agent can occur. A variety of conditions can be used, the most preferred being conditions that closely resemble conditions found in the cytoplasm of a human cell. Features such as osmolarity, pH, temperature and the concentration of cellular extract used, can be varied to optimize the association of the protein with the binding partner.

As used herein, the term “binding partner” refers to any molecule that binds to a dendritic cell associated protein. Binding partners to any one of these proteins include, but are not limited to, small molecules, peptides, polypeptides and proteins. In one embodiment, the binding partner is a co-receptor that forms a dimer complex with the protein, such complexes being necessary for efficient signal transduction. In another embodiment, the binding partner can be a transmembrane protein or a subunit of a transmembrane protein as the proteins selectively expressed by the dendritic cell can be cell surface molecules, which transduce their signals by coupling with effector proteins on the intracellular surface of the membrane.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density-sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the dendritic cell associated protein can be attached to a nitrocellulose matrix or acrylic beads. Attachment of the protein to a solid support aids in separating peptide-binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol. Biol. 69, 171-184 or identified through the use of epitope tagged proteins or GST fusion proteins.

Alternatively, the nucleic acid molecules encoding the gene for the dendritic cell associated protein can be used in a yeast two-hybrid system. The yeast two-hybrid system has been used to identify other protein partner pairs (Alifragis et al., (1997) Proc. Natl. Acad. Sci. USA 94, 13099-13104; Dong et al., (1999) Gene 237, 421-428) and can readily be adapted to employ the nucleic acid molecules encoding the selectively expressed proteins.

The dendritic cell associated protein or the nucleic acids encoding the gene for this protein can be used on an array or microarray for high-throughput screening for agents which interact with either the nucleic acids of the invention or their corresponding proteins.

An “array” or “microarray” generally refers to a grid system which has each position or probe cell occupied by a defined nucleic acid fragments also known as oligonucleotides. The arrays themselves are sometimes referred to as “chips” or “biochips” and these terms are used interchangeably. High-density nucleic acid and protein microarrays often have thousands of probe cells in a variety of grid styles.

A typical molecular detection chip includes a substrate on which an array of recognition sites, binding sites or hybridization sites are arranged. Each site has a respective molecular receptor which binds or hybridizes with a molecule having a predetermined structure. The solid support substrates which can be used to form surface of the array or chip include organic and inorganic substrates, such as glass, polystyrenes, polyimides, silicon dioxide and silicon nitride. For direct attachment of probes to the electrodes, the electrode surface must be fabricated with materials capable of forming conjugates with the probes.

Once the array is fabricated, a sample solution is applied to the molecular detection chip and molecules in the sample bind or hybridize at one or more sites. The sites at which binding occurs are detected, and one or more molecular structures within the sample are subsequently deduced. Detection of labeled batches is a traditional detection strategy and includes radioisotope, fluorescent and biotin labels, but other options are available, including electronic signal transduction.

Polymer arrays of nucleic acid probes can be used to extract information from, for example, nucleic acid samples. These samples are exposed to the probes under conditions that permit binding. The arrays are then scanned to determine to which probes the sample molecules have interacted with the nucleic acids of the polymer array. One can obtain information by careful probe selection and using algorithms to compare patterns of interactions. For example, the method is useful in screening for a dendritic cell associated protein expressed by a subset of human Langerhans cells.

Nucleic Acid Arrays

In typical applications, a complex solution containing one or more substances to be characterized contacts a polymer array comprising nucleic acids. For example, the array is comprised of nucleic acid probes. The probes of the array can be either DNA or RNA, which may be either single-stranded or double-stranded. In a preferred embodiment of the invention, the probes are arranged (either by immobilization, typically by covalent attachment, of a presynthesized probe or by synthesis of the probe on the substrate) on the substrate or chips in lanes stretching across the chip and separated, and these lanes are in turned arranged in blocks of preferably five lanes, although blocks of other sizes will have useful application. The present invention provides individual probes, sets of probes, and arrays of probe sets on chips, in specific patterns which are used to characterize the substances in a complex mixture by producing a distinct image which is representative of the binding interactions between the probes on the chip and the substances in the complex mixture. The pattern of hybridization to the chip allows inferences to be drawn about the substances present in the complex mixture.

The substances in the complex solution will bind to the nucleic acids on the array. The substances of the complex mixture which bind to the nucleic acids of the array may include, but are not limited to, complementary nucleic acids, non-complementary nucleic acids, proteins, antibodies, oligosaccharides, etc. The types of binding may include, but are not limited to, specific and non-specific, competitive and non-competitive, allosteric, cooperative, non-cooperative, complementary and non-complementary, etc. For example, the nucleic acids of the array can bind to complementary nucleic acids in the complex mixture but can also bind in a tertiary manner, independent of base pairing, to non-complementary nucleic acids.

The nucleic acids of the array or the substances of the complex mixture may be tagged with a detectable label. The detectable label can be, for example, a luminescent label, a light scattering label or a radioactive label. Accordingly, locations at which substances interact can be identified by either determining if the signal of the label has been quenched by binding or identifying locations where the signal of the label is present in cases where the substances of the complex mixture have been labeled. Based on the locations where binding is detected, information regarding the complex mixture can be obtained.

The methods of this invention will find particular use wherever high through-put of samples is required. In particular, this invention is useful in ligand screening settings and for determining the composition of complex mixtures.

Protein Arrays

Polypeptides are an exemplary system for exploring the relationship between structure and function in biology. When the twenty naturally occurring amino acids are condensed into a polymeric molecule they form a wide variety of three-dimensional configurations, each resulting from a particular amino acid sequence and solvent condition. For example, the number of possible polypeptide configurations using the twenty naturally occurring amino acids for a polymer five amino acids long is over three million. Typical proteins are more than one-hundred amino acids in length.

In typical applications, a complex solution containing one or more substances to be characterized contacts a polymer array comprising polypeptides. The polypeptides of the invention can be prepared by classical methods known in the art, for example, by using standard solid phase techniques. The standard methods include exclusive solid phase synthesis, partial solid phase synthesis methods, fragment condensation, classical solution synthesis and recombinant DNA technology (see Merrifield, (1963) Am. Chem. Soc. 85, 2149-2152). On solid phase, the synthesis is typically commenced from the C-terminal end of the peptide using an alpha-amino protected resin. A suitable starting material can be prepared, for instance, by attaching the required alpha-amino acid to a chloromethylated resin, a hydroxy-methyl resin or a benzhydrylamine resin.

The alpha-amino protecting groups are those known to be useful in the art of stepwise synthesis of peptides. Included are acyl type protecting groups, aromatic urethane type protecting groups, aliphatic urethane protecting groups and alkyl type protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride, dimethyl formamide (DMF) mixtures.

In a preferred embodiment, the polypeptides or proteins of the array can bind to other co-receptors to form a heteroduplex on the array. In yet another embodiment, the polypeptides or proteins of the array can bind to peptides or small molecules.

These procedures can also be used to synthesize peptides in which amino acids other than the twenty naturally occurring, genetically encoded amino acids are substituted at one, two, or more positions of any of the compounds of the invention. For instance, naphthylalanine can be substituted for tryptophan, facilitating synthesis. Other synthetic amino acids that can be substituted into the peptides of the present invention include L-hydroxypropyl, L-3,4-dihydroxyphenylalanyl, d-amino acids such as L-d-hydroxylysyl and D-d-methylalanyl, L-alpha-methylalanyl and beta-amino acids. Non-naturally occurring synthetic amino acids can also be incorporated into the peptides of the present invention (see Roberts et al., (1983) Peptide Synthesis 5, 341-449).

One can replace the naturally occurring side chains of the twenty genetically encoded amino acids (or D amino acids) with other side chains, for instance with groups such as alkyl, lower alkyl, cyclic four, five, six, to seven-membered alkyl, amide, amide lower alkyl, amide di(lower alkyl), lower alkoxy, hydroxy, carboxy and the lower ester derivatives thereof, and with four, five, six, to seven-membered heterocyclic. In particular, proline analogs in which the ring size of the proline residue is changed from five members to four, six or seven members can be employed. Cyclic groups can be saturated or unsaturated, and if unsaturated, can be aromatic or non-aromatic. Heterocyclic groups preferably contain one or more nitrogen, oxygen and sulphur heteroatoms. Examples of such groups include the furazanyl, furyl, imidazolidinyl, imidazolyl, imidazolinyl, isothiazolyl, isoxazolyl, morpholinyl, oxazolyl, piperazinyl, piperidyl, pyranyl, pyrazinyl, pyrazolidinyl, pyrazolinyl, pyrazolyl, pyridazinyl, pyridyl, pyrimidinyl, pyrrolidinyl, pyrrolinyl, pyrrolyl, thiadiazolyl, thiazolyl, thienyl, thiomorpholinyl and triazolyl. These heterocyclic groups can be substituted or unsubstituted. Where a group is substituted, the substituent can be alkyl, alkoxy, halogen, oxygen, or substituted or unsubstituted phenyl.

One can also readily modify the peptides of the instant invention by phosphorylation (see Bannwarth et al., (1996) Biorg. Med. Chem. Let. 6, 2141-2146) and other methods for making peptide derivatives of the compounds of the present invention are described in Hruby et al., (1990) Biochem. J. 268, 249-262). Thus, the peptide compounds of the invention also serve as a basis to prepare peptide mimetics with similar biological activity. The array can also comprise peptide mimetics with the same or similar desired biological activity as the corresponding peptide compound but with more favorable activity than the peptide with respect to solubility, stability, and susceptibility to hydrolysis and proteolysis (see Morgan et al., (1989) Ann. Rep. Med. Chem. 24, 243-252).

Peptides suitable for use in this embodiment generally include those peptides, for example, ligands, that bind to a receptor, such as membrane proteins. Such peptides typically comprise about 150 amino acid residues or less and, more preferably, about 100 amino acid residues or less. Polypeptides or proteins suitable for use in this embodiment generally include those polypeptides or proteins that interact with a receptor, such as a co-receptor or G protein. Such polypeptides or proteins typically comprise about 150 amino acid residues or more and, more preferably, about 400 amino acids or more.

The peptides of the present invention may exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues may also be substituted with a homocysteine. Other embodiments of this invention provide for analogs of these disulfide derivatives in which one of the sulfurs has been replaced by a CH2 group or other isostere for sulfur. These analogs can be made via an intramolecular or intermolecular displacement, using methods known in the art.

Methods to Identify Agents that Modulate Expression

Another embodiment of the present invention provides methods for identifying agents that modulate the expression of a nucleic acid encoding a dendritic cell associated protein, preferably one expressed by Langerhans cells. In a more preferred embodiment, the nucleic acid can encode a dendritic cell associated protein expressed by a subset or subtype of Langerhans cells. Such assays may utilize any available means of monitoring for changes in the expression level of the nucleic acids. As used herein, an agent is said to modulate the expression of a nucleic acid, if it is capable of up- or down-regulating expression of the nucleic acid in a cell.

In one assay format, cell lines that contain reporter gene fusions between the open reading frame of any one of the nucleic acids encoding a dendritic cell associated protein and any assay fusion partner may be prepared. Numerous assay fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding at least one of the dendritic cell associated proteins.

Additional assay formats may be used to monitor the ability of the agent to modulate the expression of a nucleic acid encoding at least one dendritic cell associated protein. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press.

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementary nucleotides which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1989) Molecular Cloning—A Laboratory Manual, Cold Spring Harbor Laboratory Press; or Ausubel et al., (1998) Current Protocols in Molecular Biology, Greene Publishing Company.

Hybridization conditions are modified using known methods, such as those described by Sambrook et al., (1989) and Ausubel et al., (1998) as required for each probe. Hybridization of total cellular RNA or RNA enriched for polyA+ RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass wafer can then be exposed to total cellular RNA or polyA RNA from a sample under conditions in which the affixed sequences will specifically hybridize. Such glass wafers and hybridization methods are widely available, for example, those disclosed by Beattie, (1995) WO 95/11755.

Hybridization for qualitative and quantitative analysis of mRNA may also be carried out by using a RNase Protection Assay (i.e., RPA, see Ma et al., (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea-polyacrylamide gels for analysis.

In another assay format, agents which effect the expression of the instant gene products, cells or cell lines would first be identified which express said gene products physiologically. Cells and cell lines so identified would be expected to comprise the necessary cellular machinery such that the fidelity of modulation of the transcriptional apparatus is maintained with regard to exogenous contact of agent with appropriate surface transduction mechanisms and the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art (e.g., Sambrook et al., (1989) Molecular Cloning—A Laboratory Manual, Cold Spring Harbor Laboratory Press).

Cells or cell lines transduced or transfected as outlined above would then be contacted with agents under appropriate conditions; for example, the agent comprises an acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides from disrupted cells are fractionated such that a polypeptide fraction is pooled and contacted with an antibody to be further processed by immunological assay (e.g., ELISA, immunoprecipitation or Western blot). The pool of proteins isolated from the “agent contacted” sample will be compared with a control sample where only the excipient is contacted with the cells and an increase or decrease in the immunologically generated signal from the agent contacted sample compared to the control will be used to distinguish the effectiveness of the agent.

Methods to Identify Agents That Modulate Activity

Another embodiment of the present invention provides methods for identifying agents that modulate at least one activity of a dendritic cell associated protein expressed by human dendritic cells, preferably Langerhans cells, even more preferably, a subset or subtype of Langerhans cells. Such methods or assays may utilize any means of monitoring or detecting the desired activity including, but not limited to, biochemical and electrophysiological methods.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site and its conformation in connection with the agent's action. Agents can be rationally selected or rationally designed by utilizing the peptide sequences to identify proposed binding motifs, glycosylation and phosphorylation sites on the protein.

The agents of the present invention can be, as examples, peptides, small molecules, antibodies, vitamin derivatives, as well as carbohydrates. A skilled artisan can readily recognize that there is no limit as to the structural nature of the agents. Dominant-negative proteins, DNA encoding these proteins, antibodies to these proteins, peptide fragments of these proteins or mimics of these proteins may be contacted with cells to affect function. “Mimic” as used herein refers to the modification of a region or several regions of a peptide molecule to provide a structure chemically different from the parent peptide but topographically and functionally similar to the parent peptide (see Meyers, (1995) Molecular Biology & Biotechnology, VCH Publishers).

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

### EXAMPLES

### Example 1

### Purification of CD34⁺ Stem Cells From Leukapheresis Products

Cancer patients who had undergone chemotherapy followed by G-CSF treatment in preparation for autologous stem cell transplantation (or healthy donors mobilized with G-CSF alone) were selected on the basis of how efficiently CD34⁺ stem cells were mobilized into their peripheral blood. Typically, patients with ≧1% CD34⁺ peripheral blood mononuclear cells were asked to donate either a part of a standard (60 ml) leukapheresis or to undergo a separate apheresis procedure for subsequent donation. While cancer patients were used in this experiment, healthy donors can also be employed. Informed consent was obtained from all patients, and the protocol was approved by the Yale University School of Medicine Human Investigational Studies Committee. CD34⁺ stem cells were immunomagnetically purified from leukapheresis products with either a midi-MACS system (CD34 progenitor cell isolation kit or multi sort kit) following the protocol of the manufacturer (Miltenyi Biotec) or a Baxter Isolex device. Yields of CD34⁺ cells varied from 1×10⁷ to 2.5×10⁸/ml leukapheresis; purity ranged from 85 to 95% CD34⁺ after a single selection. CD34⁺ cells were frozen in aliquots of 2.5×10⁶ in PBS supplemented with 20% human albumin and 10% DMSO and stored in liquid nitrogen.

### Example 2

### Culture Conditions

Cells were thawed and cultured at 1×10⁴cells/ml/well in 24-well plates in media prepared exactly as described by Strobl et al., (1997) Blood 90, 1425-1434. Specifically, cells were grown in X-VIVO 15 containing 100 ng/ml GM-CSF (5.6 IU/mg), 20 ng/ml stem cell factor (5×10⁴ U/mg), 2.5 ng/ml TNF-alpha (2×10⁷ U/mg), 0.5 ng/ml TGF-beta-1 (2×10⁷ U/mg) and 100 ng/ml Flt3 ligand (Flt3L). All cytokines were purchased from PeproTech, except for GM-CSF and Flt3L, which were obtained from Immunex. Cultures were incubated at 37° C. with 5% CO₂ in a humidified environment for seven to ten days without feeding or replating; by this time total cell number had increased by 50- to 100-fold. For maturation, X-VIVO+1× cytokines was supplemented with additional 10 ng/ml of TNF-alpha or with 250 ng/ml of LPS (Sigma).

### Example 3

### Antibodies and Flow Cytometry

Antibodies to the following proteins were used: CD1a-PE (clone BL6), CD83-PE (Immunotech); CD1a-FITC (clone HI149), CD14-FITC, CD66b-FITC, CD86-PE, CD107a, HLA-DR-FITC, HLA-DR-CyChrome, (PharMingen); HLA-A,B,C-FITC (Clone W6-32, Biode-sign International); CLA 1 (American Type Culture Collection clone HECA-452). LFA-3 Antibodies were obtained from mouse ascites. Fluorescence microscopy was performed as described in Pierre et al., (1997) Nature 388, 787-792. Flow cytometry was performed by standard procedure. When detection of intracellular protein was required, cells were previously fixed and premeabilized as described in Inaba et al., (1997) J. Exp. Med. 186, 665-672.

### Example 4

### Generation of Dendritic Cell Precursors

Before autologous stem cell transplantation, cancer patients are typically treated with G-CSF to mobilize large numbers of progenitor cells into the peripheral blood. Given the quantity and availability of these preparations from patients and normal volunteers, their utility as a source of dendritic cells following in vitro differentiation was investigated using a modification of techniques previously described for the differentiation of CD34⁺ cells from cord blood. CD34⁺ cells from leukapheresis product were immunopurified to >95% homogeneity and then seeded in twenty-four well plates at 1×10⁴ cells/well in serum-free medium (X-VIVO 15) containing GM-CSF, TNF-alpha, TGF-beta, SCF and Flt3L. Cells were grown for seven to ten days without further manipulation.

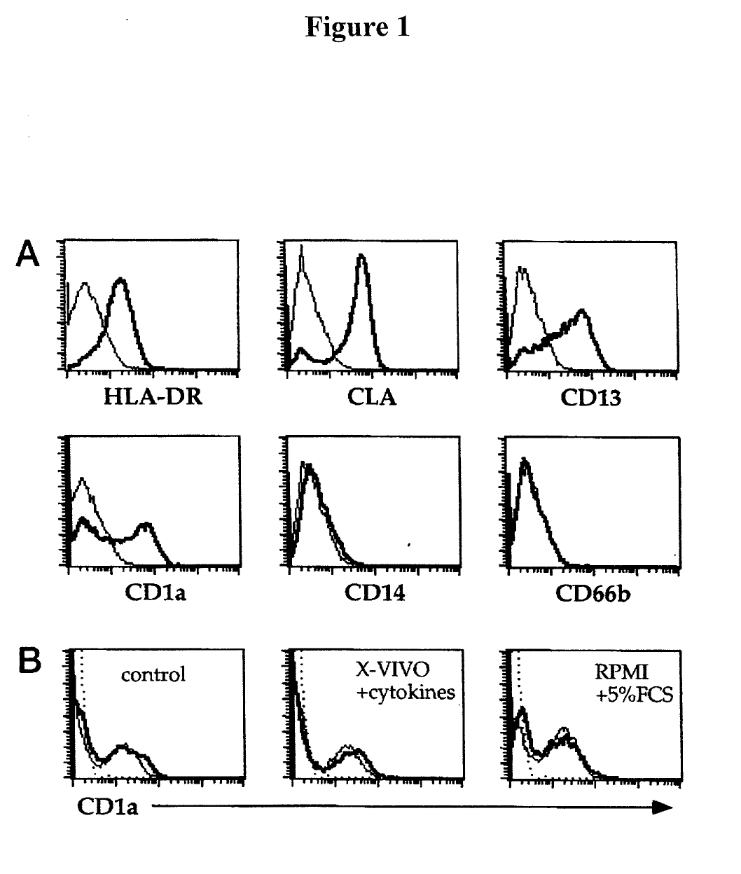

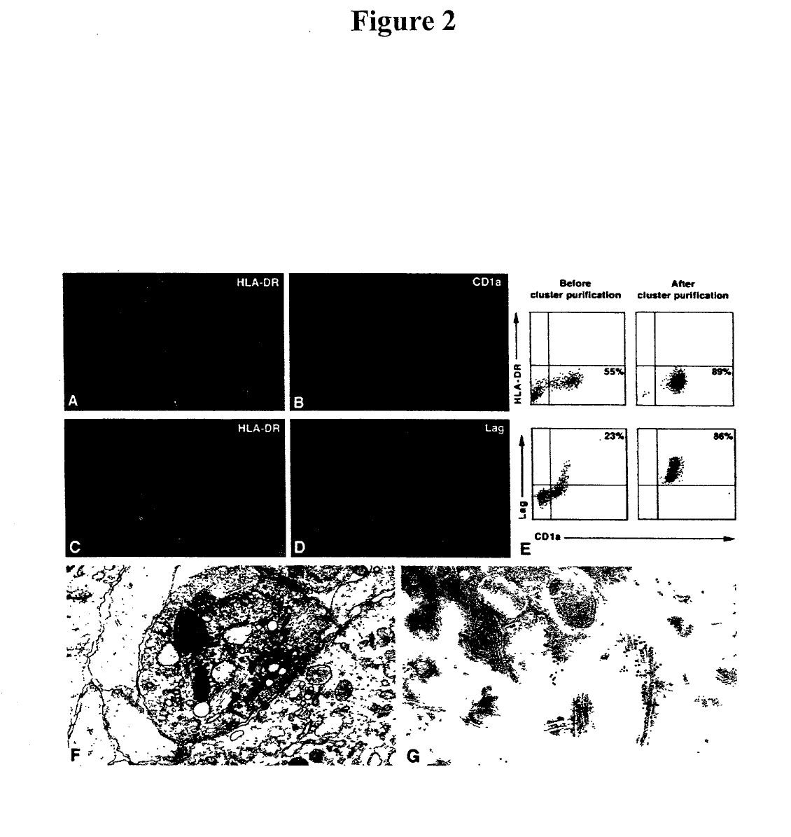

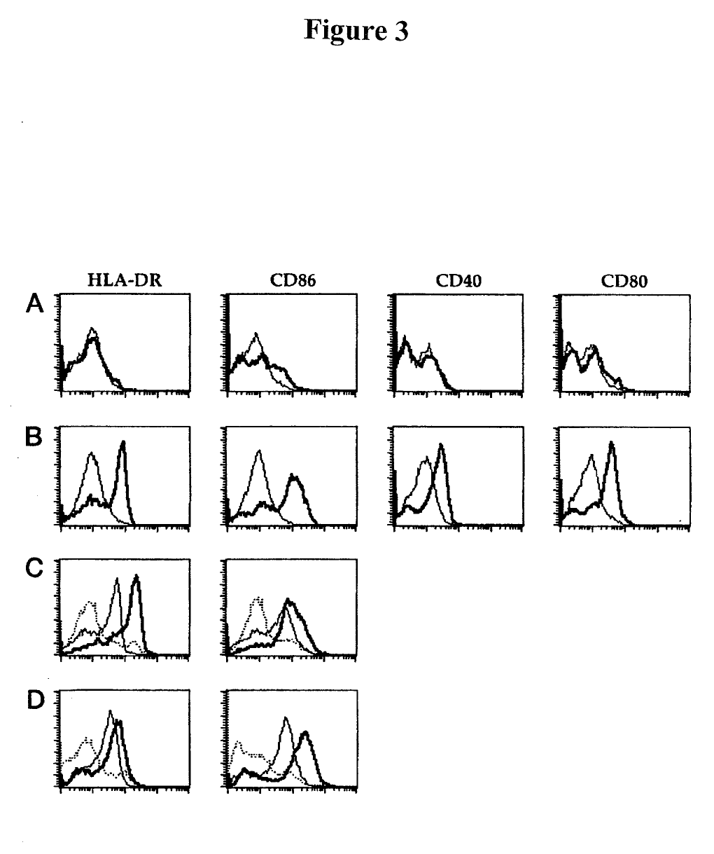

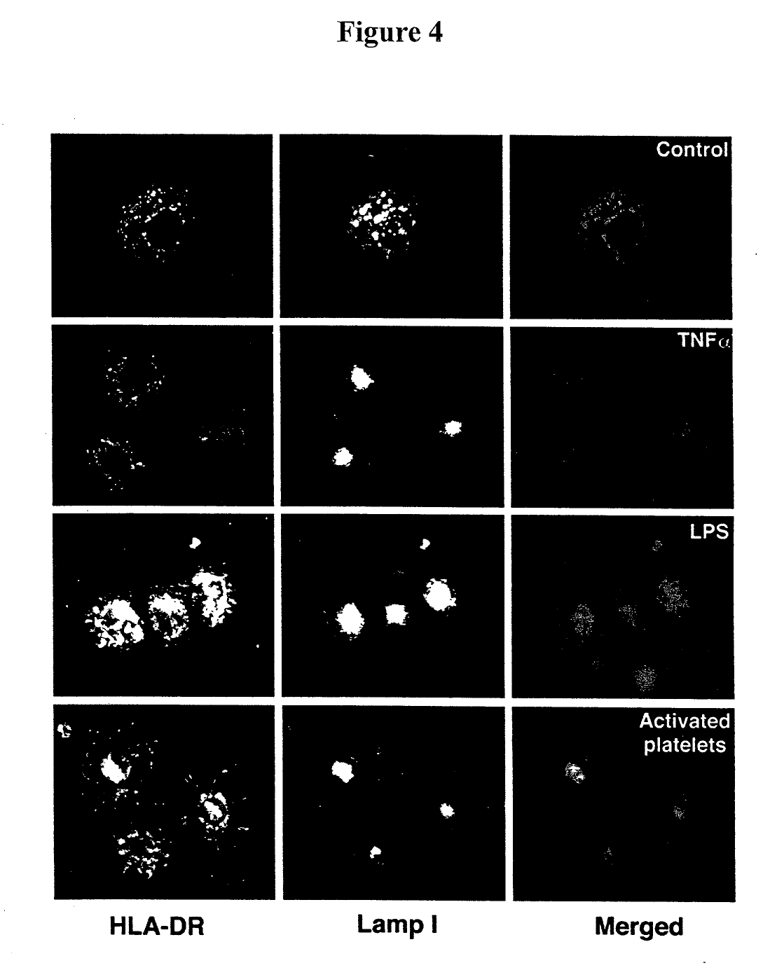

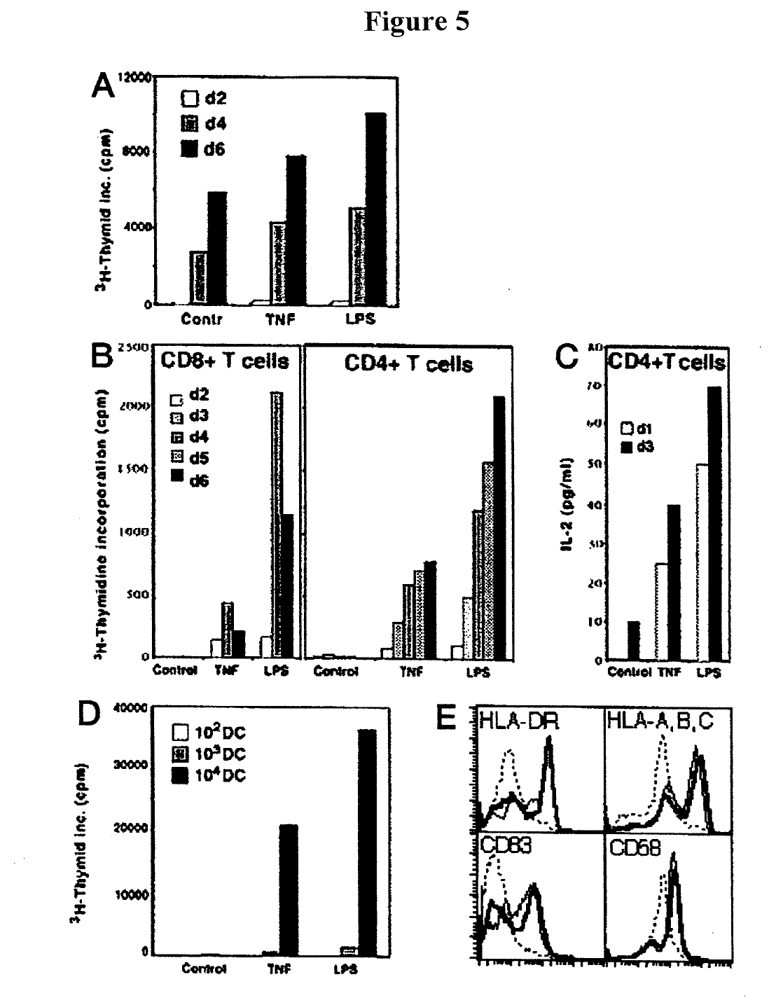

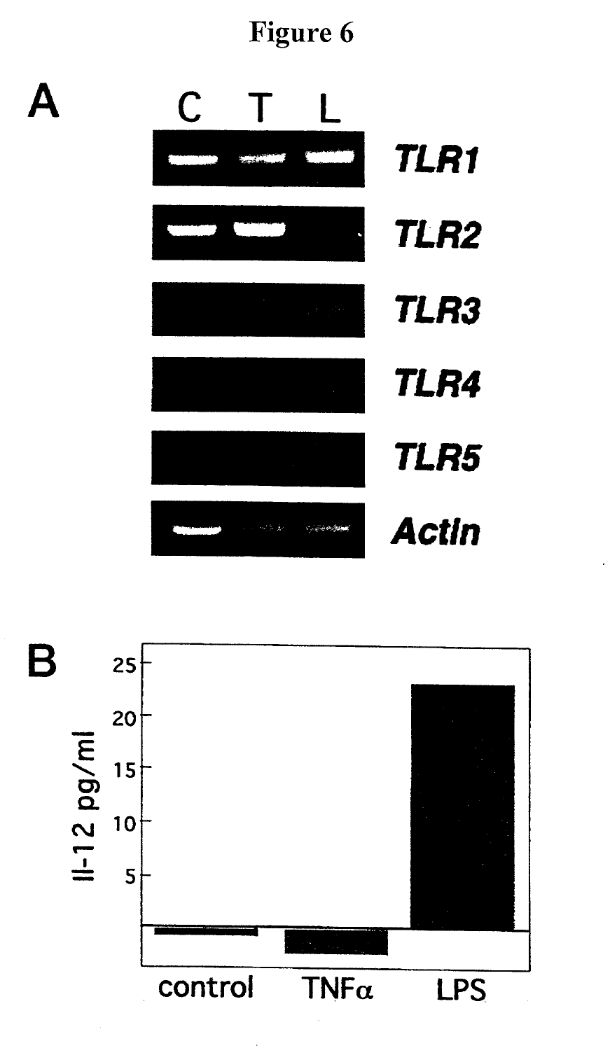

By day seven, total cell number had increased fifty to one-hundred fold with 35-60% of the cells being positive for CD1a. A representative profile of cell-surface expression for various markers at day eight is shown in FIG. 1A. Interestingly, and in contrast with the majority of other methods for producing dendritic cells from CD34⁺ precursors, most of the cells in culture were HLA-DR^{dim} as expected for immature dendritic cells. No monocyte- or granulocyte-related cells were detected in culture as indicated by the absence of CD14⁺ and CD66b⁺ cells; these markers were also not detected at earlier times of culture (days two through six, data not shown), suggesting that there was not even transient expression of early granulocyte markers. The majority of the cells were positive for CD13, suggesting that they were myeloid in origin. Interestingly, most of the cells were CLA⁺, a feature consistent with their being Langerhans cell-committed precursors.

An important feature of the CD1a⁺/CLA⁺cells (presumptive Langerhans cells) was their phenotypic stability. As shown in FIG. 1B, CD1a expression did not decrease with time in culture when day ten cells were replated either in serum-free X-VIVO or in RPMI 1640 containing 5% fetal calf serum. However, the serum-fed cells exhibited a marked increase in surface HLA-DR (data not shown), suggesting that maturation had been induced by a cytokine or LPS contaminant in the serum. In any event, it is clear that the presumptive Langerhans cells could be maintained and apparently in an immature state, for days if grown in a defined serum-free medium (see below).

### Example 5

### Maintenance of Presumptive Langerhans Cells in an Immature State

A more detailed analysis of dendritic cells generated from the mobilized CD34⁺ precursors was performed next. Specifically, it was desirable to determine whether homogenous populations of presumptive Langerhans cell precursors could be enriched from the cultures, maintained in an immature dendritic cell phenotype and then triggered to mature in a synchronous fashion. From day four on, loosely adherent aggregates of cells appeared in culture, which increased in size and number with time. At day seven, presumptive Langerhans cells were found only in multicellular clusters, as indicated by staining with CD1a and with the unique Langerhans cell marker Lag (FIGS. 2A-D). The cluster cells also exhibited most of their MHC class II intracellularly with very little on the plasma membrane, typical of immature dendritic cells. These features were highly reminiscent of the dendritic cells produced from mouse bone marrow in which proliferating and differentiating dendritic cells accumulate as immature dendritic cells in analogous cell clusters.

To enrich for CD1a⁺ cells, the clusters were purified by 1×g sedimentation on 7.5% BSA columns. Clusters were purified by gently harvesting cells with a pipette and layering on top of six ml of 7.5% BSA (Sigma) in 15 ml tubes; up to eight wells were loaded per column. No adherent cells remained in the wells upon harvesting. After thirty minutes on ice, single cells in suspension were removed by aspirating the BSA columns until 3.5 ml remained. Clusters were concentrated by centrifugation at 300×g, resuspended in growth media, and cultured at 5×10⁵ cells/ml/well in 24-well plates for an additional two days.

FACS analysis revealed that this procedure produced excellent yields both in term of number of cells and purity (70-90% CD1a 1/HLA-DR^{dim}) (FIG. 2E). The Langerhans cell marker Lag was expressed by >70% of the cluster-purified cells. Cells were also examined by electron microscopy for the presence of pathognomonic Langerhans cell-specific organelles.

For conventional plastic sections, cells were fixed with 2.5% glutaraldehyde in 100 mM cacodylate, pH 7.4, for one hour at room temperature, washed once with 100 mM cacodylate, treated with 2% OsO₄ for one hour, treated with 1% uranyl acetate in 50 mM maleate buffer (pH 5.2) for one hour, dehydrated using a graded ethanol series and acetone, and pelleted before embedding in Epon and sectioning. For protein A gold labeling of Lag-stained cryosections, cells were fixed in 4% paraformaldehyde (PFA) in 100 mM Hepes (pH 7.4) for one hour at room temperature and then processed exactly as per Sodeik et al., (1997) J. Cell Biol. 136, 1007-1021.

Electron microscopy of the purified cells revealed the abundant presence of pathognomonic Langerhans cell-specific organelles, Birbeck granules (FIG. 2F, arrows). The granules persisted for several days in immature Langerhans cell in culture (data not shown). Birbeck granules were found by protein A-gold immunocytochemistry of cryosections to be specifically labeled by anti-Lag Antibodies (FIG. 2G) but negative for MHC class II or Lamp 1 (not shown).

### Example 6

### Generation of Mature Langerhans Cells

If left undisturbed, cells in the proliferating clusters exhibited this immature Langerhans cell phenotype for up to twelve days. However, upon disruption of the clusters by vigorous repeated pipetting, the cells appeared to mature as indicated by increased surface expression of MHC class II and other markers of mature dendritic cells (CD86, CD40, CD80) (FIG. 3B). Yet, when clusters were purified and gently re-plated in fresh growth medium (X-VIVO plus cytokines), the immature phenotype was maintained and spontaneous maturation was not observed, as indicated by the low level of HLA-DR and co-stimulatory molecules expressed on the cell surface (FIG. 3A). Previous attempts to cultivate human dendritic cells from CD34⁺ precursors have yielded only mature cells with high levels of cell-surface MHC class II products and costimulatory molecules. Conceivably, this difference may reflect the sensitivity of dendritic cells to physical manipulation, which might disrupt and activate cell clusters.

Based on their surface markers and abundance of intracellular Birbeck granules, the cells were indistinguishable from epidermal Langerhans cells. The cells were not only irreversibly committed to the Langerhans cell lineage, but also arrested in an immature state (low surface MHC class II and accessory molecules) unless activated by proinflammatory mediators or physical disruption.

Maturation was induced by three different mediators TNF-alpha, LPS and CD40L. Each of these agents generated mature cells that were similar on the basis of their surface marker profiles as determined by flow cytometry. However, several striking differences were uncovered by more detailed cell biological and functional analysis. By confocal microscopy, each agent was found to produce cells exhibiting markedly different morphologies. Perhaps most important was the finding that TNF-alpha treatment drove the cells no further than the “intermediate” dendritic cell phenotype, characterized by an accumulation of non-lysosomal intracellular vesicles that, in mouse bone marrow-derived dendritic cells, carry peptide-loaded MHC molecules to the plasma membrane. Thus, TNF-alpha may be capable of inducing only the earliest steps in dendritic cell maturation. This is in contrast to LPS and CD40L that can convert immature cells, which typically localize the bulk of the MHC class II products in late endosomes and lysosomes, to mature cells that express little if any intracellular class II.

The phenotypic differences observed were reflected, at least in part, at a functional level. Using an allogeneic response as an assay, diminished T cell stimulatory activity was consistently observed when TNF-alpha treated rather then LPS-treated dendritic cells were used as antigen processing cells. Applicants also observed that the important Th1-driving cytokine, IL-12, was produced only by LPS-treated dendritic cells and not by TNF-alpha treated cells.

In addition, the present invention for Langerhans cell production from mobilized CD34⁺ cells has clinical applications. Not only can large numbers of autologous cells be produced with reasonable ease, but the fact that the Langerhans cells generated remain immature in culture will allow them to remain in the state best suited for antigen accumulation. Also relevant to any potential clinical applications was the fact that once differentiated, the Langerhans cells produced are phenotypically stable. To be maximally effective, dendritic cells must maintain their differentiated features upon reintroduction into the blood stream or tissues, environments in which the cytokine composition is markedly different from the medium in which they had been cultured. Dendritic cells differentiated from peripheral blood monocytes, for example, may rapidly lose their dendritic cell features upon removal of IL-4 or GM-CSF, suggesting that they may not resemble dendritic cells for long after in vivo reintroduction. The in vitro stability of the CD34-derived Langerhans cells described here indicates that they may have critical advantages as potential immunotherapeutic vehicles.

### Example 7

### Generation of Mature Langerhans Cell Subtypes

The controlled maturation of gently purified cluster Langerhans cells could be accomplished by using proinflammatory agents such as TNF-alpha, LPS or CD40L. Fixed, activated human platelets were used as a source of membrane-bound CD40L. After two days, FACS analysis demonstrated that these agents greatly enhanced the expression of HLA-DR, costimulatory molecules, and CD83 on the plasma membrane (FIG. 3, C and D and FIG. 5E).

Thus, as previously shown for monocyte-derived dendritic cells, epidermal Langerhans cells, and bone marrow-derived dendritic cells, treatment with TNF-alpha, LPS or CD40L induced a terminal dendritic cell differentiation from an immature to a mature phenotype. Although each of these treatments induced qualitatively similar differences when assayed by FACS, analysis by confocal microscopy revealed significant differences among the differently treated cells, in terms of both morphology and maturation stage. Cells from purified clusters, replated in regular growth medium, exhibited the phenotype typical of early dendritic cells: class II is intracellularly distributed and perfectly colocalized with lysosomal markers (FIG. 4, top row) (Pierre et al., (1997) Nature 388, 787-792). Following TNF-alpha treatment (12.5 ng/ml), nearly all of the cells exhibited the “intermediate” phenotype previously observed only in rat and mouse dendritic cells. The intermediate dendritic cells were characterized by the accumulation of MHC class II on the surface as well as in peripheral intracellular vesicles (class II vesicles or “CIIV”) devoid of lysosomal markers (FIG. 4, second row). The lysosomes were largely depleted of MHC class II and clustered in the perinuclear cytoplasm. The accumulation of cells in this intermediate phenotype suggests a role for TNF-alpha in the early but not the late events of dendritic cell or Langerhans cell activation. Thus, TNF-alpha may be unable to drive the maturation process to completion.

LPS-treated Langerhans cells were also extremely homogeneous in morphology, but their appearance was quite different from TNF-alpha treated cells. The maturation driven by LPS seemed to be more advanced; the cells appeared smaller, but with abundant phyllopodia and more of the MHC class II at the plasma membrane than in the TNF-alpha treated cells (FIG. 4, third row). Therefore, the LPS-treated cells were reminiscent of the mature or “late” stage identified in the murine system, although the structure of the lysosomal compartment in LPS-treated human dendritic cells was much less compact and indeed more similar to what was observed in intermediate cells.

Activated platelets, a source of CD40L, induced the most dramatic change in cell morphology (FIG. 4, bottom row). Gel-purified platelets were prepared, activated and fixed exactly as per Tuszynski et al., (1987) Mod. Methods Pharmacol. 4, 267-200. A total of 1.2×10⁸ platelets/well were added to 24-well plates, spun at 1200×g, and washed two times with PBS. Cluster-purified dendritic cells were then plated on top of these platelet lawns. Again, virtually all of the MHC class II was found at the plasma membrane, which was now organized into long dendrites. Class II-negative lysosomes were tightly clustered in the perinuclear region. Importantly, these mature phenotypes were stable, with the Langerhans cells expressing high levels of surface HLA-DR and maintaining CD1a for days (data not shown). Indeed, simply plating clusters in RPMI 1640 supplemented only with fetal calf serum, as opposed to cytokine-supplemented X-VIVO, supported Langerhans cell maturation. Together, these findings indicate that Langerhans cells prepared in this way are irreversibly committed to a dendritic cell phenotype.

### Example 8

### T Cell Stimulation by Mature Langerhans Cells

Differences in morphology and cell organization were compared to differences in function to determine if a correlation exists. Specifically, the T cell stimulatory capacity of the differently treated dendritic cells in an alloreaction using CD8⁺ T cells was examined.

For T cell isolation, peripheral blood mononuclear cells were obtained by leukapheresis from adult volunteer donors and further purified by centrifugation over Lymphocyte Separation Medium (Organon Teknika) according to the manufacturer's instructions. Isolated peripheral blood mononuclear cells were washed three times in HBSS (Mg²⁺ and Ca²⁺ free) and either used immediately or suspended in 10% DMSO and 90% heat-inactivated fetal calf serum and cryopreserved in liquid nitrogen. No differences were seen in the responses of cells recovered from cryopreservation compared with freshly isolated cells. CD4⁺ and CD8⁺ T cells were isolated as described respectively in Ma & Pober (1998) J. Immunol. 161, 2158-2167 and Biedermann & Pober (1998) J. Immunol. 161, 4679-4687.

Alloreactions were set up in round-bottom 96-well plates in triplicate. Unless otherwise indicated, 300,000 purified CD4⁺ T cells or 150,000 purified CD8⁺ T cells were added to 1,000 fixed dendritic cells. During the last eighteen to twenty-four hours of co-culture on the indicated days, 1 mCi [³H]thymidine (NEN Life Science Products) was added to each well, and proliferation was assessed by [³H]thymidine incorporation. The plates were harvested with a 96-well harvester and counted on a Microbeta scintillation counter (Wallac). For IL-2 detection in alloreaction, antibody to IL-2 receptor gamma-chain (anti-TAC, IgG1, used at 20 mg/ml) was added to prevent cytokine utilization. Supernatant was collected on day one or three and assayed for IL-2 by ELISA.

Dendritic cells were cluster purified at day seven and replated in regular growth media or in the presence of LPS or TNF-alpha, on day nine dendritic cells were mixed with CD8⁺ T cells. After two to six days, cells were harvested, washed extensively, and proliferation was evaluated. As shown in FIG. 5A, LPS-treated dendritic cells were the most efficient in stimulating CD8⁺ T cell proliferation when compared with control or TNF-alpha treated dendritic cells, although the differences were not very pronounced. In fact, even control dendritic cells (i.e., cells not treated with TNF-alpha or LPS before assay) were able to support a T cell response. Interaction with T cells alone is almost certainly capable of inducing dendritic cell maturation due to the presence of CD40L on T cells. Thus, it was likely that maturation of even the untreated control dendritic cells occurred during the course of the proliferation assay, obscuring any possible differences in stimulatory capacity. To circumvent this problem, we decided to use for the assay previously fixed dendritic cells to prevent further maturation due to T cell contact.

As before, control, TNF-alpha and LPS-treated dendritic cell were harvested, but fixed with 0.5% PFA and washed extensively, before co-culture with freshly isolated CD4⁺ and CD8⁺ T cells. Although the magnitude of the T cell proliferative response was markedly lower using fixed dendritic cells, the results clearly indicated that both CD4⁺ and CD8⁺ responses were completely dependent on prior exposure of the dendritic cells to a maturational stimulus with LPS-treated dendritic cells being the most efficient (FIG. 5B). Similar results were obtained when IL-2 production by CD4⁺ cells was assayed (FIG. 5C). Despite the differences in stimulatory capacity between TNF-alpha and LPS-treated dendritic cells, both treatments caused equivalent up-regulation of MHC class I, MHC class II, LFA-3 (CD58) and the dendritic cell-maturation marker CD83 (FIG. 5E). Although just 10% of the total T cell stimulatory activity was left upon dendritic cell fixation, the magnitude of these responses was still clearly dependent on the number of dendritic cells added to the assay (FIG. 5D).

The T cell stimulatory activity of human dendritic cell seems to be much more affected by PFA fixation than what has previously been shown for mouse dendritic cells. While this difference may reflect differences between the mouse and human dendritic cells used, it should also be noted that the murine dendritic cell studies were conducted using T cell hybridomas as a read out, as opposed to the primary T cells used here. One interesting feature of our culture system is that the cells are sensitive to LPS, although they do not express CD14, generally thought to be an important LPS receptor. Because the cells were grown in serum-free media, another potential mediator of LPS responsiveness, LPS binding protein, was also unlikely to play a role.

### Example 9

### Toll-Like Receptor Expression in Mature Langerhans Cell Subtypes

Recently, a new family of molecules involved in the response to LPS, the Toll-like receptors (TLR1 through TLR5) has been cloned and partially characterized. Therefore, Toll-like receptor expression by in vitro derived Langerhans cells was examined along with expression levels to determine if it was affected by maturation.

Using specific oligonucleotide primers for each of the human TLR molecules, RT-PCR was performed on cDNA obtained from cluster-purified cells treated for forty-eight hours with or without TNF-alpha or LPS. For the RT-PCR assay, total RNA was isolated from differently treated dendritic cell using Trizol® (Life Technologies) and 0.5 mg was used for reverse transcription using avain myeloblastosis virus reverse transcriptase (Life Technologies). Primer sequences were as follows: TLR1,

(SEQ ID NO: 1)

| | |
|---|---|
| TLR1, | 5′-CCTGGCAAGAGCATTGTGGAA and |

(SEQ ID NO: 2)

3′-TGTAATCTATTTCTTTGCTTGCTCTGTCAG;

(SEQ ID NO: 3)

| | |
|---|---|
| TLR2, | 5′-GTGAAGAGTGAGTGGTGCAAGTAT and |

(SEQ ID NO: 4)

3′-CATAAAGAT CCCAACTAGACAAAGACTGG;

(SEQ ID NO: 5)

| | |
|---|---|
| TLR3, | 5′-TGGGTCTGGGAACATTTCTCTTCA and |

(SEQ ID NO: 6)

3′-ATAAATTTAATGTACAGAGTTTTTGGATCC;

(SEQ ID NO: 7)

| | |
|---|---|
| TLR4, | 5′-CTGAGCAGTCGTGCTGGTATCATC and |

(SEQ ID NO: 8)

3′-ACCCAGCTGGGCAAGAAATGCCTCAGGAGG; and

(SEQ ID NO: 9)

| | |
|---|---|
| TLR5, | 5′-GCCCAGGGCAGGTGCTTATCT and |

(SEQ ID NO: 10)

3′-GATAACTTGGTGCAAATACAAAGTGAAGA.

As shown in FIG. 6A, distinct expression patterns were observed for the five receptors depending on the type of inflammatory agent used. While the expression of TLR1 mRNA was almost unchanged regardless of whether TNF-alpha or LPS were added to the cultures, LPS but not TNF-alpha induced a dramatic down-regulation of TLR2. Conversely, expression of TLR3 and TLR4 were up-regulated to varying extents by both mediators, but more so by LPS than by TNF-alpha. Thus, TNF-alpha and LPS exert markedly different effects on the expression of individual members of the TLR family, further emphasizing that different mediators can elicit qualitatively different types of dendritic cell maturation.

It was also determined that the cells were markedly different in another feature of likely functional importance, namely the expression of Toll-like receptor family members. Recent evidence strongly suggests that Toll-like receptors play critical roles in innate immunity by providing a means whereby cells of the innate immune system distinguish between self and invading microorganisms. LPS is thought to bind and activate at least two members of this family, TLR2 and TLR4. Thus, the expression of these two receptors and their relatives can be expected to play an important role in dendritic cell function. Interestingly, it was determined that LPS and TNF-alpha elicited markedly different patterns of TLR gene expression following induction of dendritic cell maturation. In particular, LPS induced the complete down-regulation of mRNA encoding one of its presumptive receptors TLR2, while TNF-alpha had no effect on TLR2 expression.

On the contrary, TLR4 is completely absent in immature cells, and its expression is differentially induced upon maturation. That LPS induced higher levels of TLR3 and TLR4 than did TNF-alpha implies a further qualitative difference in the effects of these mediators, but also may reflect the ability of LPS to drive maturation to “completion” or at least beyond the intermediate phenotype elicited by TNF-alpha.

### Example 10

### Cytokine Production in Mature Langerhans Cell Subtypes

Finally, cytokine production by the Langerhans cells was examined to determine if it was differentially regulated depending on the nature of the maturational stimulus. IL-12 production was assayed in cell-culture supernatants by ELISA for p70 antigen using a kit from Endogen. Only LPS-matured, but not TNF-alpha matured, dendritic cells produced significant levels of IL-12 (FIG. 6B).

### Example 11

### Identification of a Dendritic Cell Associated Protein

Dendritic cells exhibit a pattern of activation resulting in their conversion from immature cells specialized for antigen accumulation to mature cells specialized for T cell stimulation. Recently, the inventors have developed a new dendritic cell culture system which provides for methods of producing large numbers of mature dendritic cells suitable for use in the novel methods of the present invention (Gatti et al., (2000) J. Immunol. 164, 3600-3607 herein incorporated by reference in its entirety). Starting with leukapheresis products from G-CSF-mobilized human donors, immunomagnetically purified CD34⁺ precursor cells were isolated. The procedures employed provided well characterized and highly functional populations of dendritic cells. These cells were induced to activate by treatment with TNF-alpha. Proteomic analyses of immature and mature activated populations of dendritic cells were carried out to identify proteins selectively expressed by these activated cells.

FACS analysis demonstrated that TNF-alpha treatment greatly enhanced the expression of HLA-DR, co-stimulatory molecules and CD83 on the plasma membrane. Thus, as previously shown for monocyte derived dendritic cells, treatment with TNF-alpha induced a dendritic cell differentiation from an immature to a mature phenotype.

The T cell stimulatory capacity of the TNF-alpha treated dendritic cells was tested in an alloreaction, using CD8⁺ T cells. Dendritic cell s were cluster purified at day seven and replated in regular growth media or in the presence TNF-alpha; on day nine dendritic cells were mixed with CD8⁺ T cells. After two to six days, cells were harvested, washed extensively, and proliferation was evaluated. TNF-alpha treated dendritic cell were able to stimulate the CD8⁺ T cells better than non-treated dendritic cells.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

What is claimed:

1. A method of isolating a dendritic cell associated protein comprising:
(a) treating a dendritic cell with a first agent which alters the expression or activity of a protein associated with the dendritic cell;
(b) isolating a cell extract from the dendritic cell;
(c) associating the extract with at least one second agent;
(d) detecting the binding of the second agent to the dendritic cell associated protein in the extract; and
(e) isolating the dendritic cell associated protein bound to the second agent from the extract.

2. The method of claim 1 wherein the first agent is selected from the group consisting of proteins and nucleic acids.

3. The method of claim 2 wherein the protein is a cytokine.

4. The method of claim 2 wherein the protein is a pathogen isolated from a microorganism selected from the group consisting of viruses, bacteria, fungi and parasites.

5. The method of claim 2 wherein the protein is selected from the group consisting of interleukin-1beta, prostaglandin-E2, tumor necrosis factor-alpha, lipopolysaccharide and CD40 ligand.

6. The method of claim 2 wherein the nucleic acid is selected from the group consisting of double stranded RNA and CpG DNA.

7. The method of claim 1 wherein the cell extract is isolated from a cell component selected from the group consisting of the nucleus, cytosol and cell membrane.

8. The method of claim 1 wherein the dendritic cell is a immature dendritic cell.

9. The method of claim 1 wherein the dendritic cell is a mature dendritic cell.

10. The method of claim 9 wherein the dendritic cell is a Langerhans cell.

11. The method of claim 10 wherein the Langerhans cell contains Birbeck granules.

12. The method of claim 1 wherein the dendritic cell is a human dendritic cell.

13. The method of claim 1 wherein the second agent is selected from the group consisting of proteins, polypeptides, peptides, macromolecules, chemical compounds, oligonucleotides and nucleic acids.

14. The method of claim 1 wherein the second agent is an antibody.

15. The method of claim 1 wherein the dendritic cell associated protein is selected from the group of proteins consisting of CD1a, CD40, CD80, CD83, CD86, HLA-DR, HLA-A, HLA-B, HLA-C, CLA1, LFA-3, TLR-1, TLR-2, TLR-3, TLR-4 and TLR-5.

16. The method of claim 1 wherein the dendritic cell associated protein in the extract is on an array.

17. The method of claim 1 wherein the second agent is on an array comprising a plurality of agents capable of binding to at least one dendritic cell associated protein present in the extract.

18. The method of claim 16 or 17 wherein the array is a biological chip.

19. A method of detecting the presence of a disease associated with altered dendritic cell activity in a patient comprising:
(a) isolating a sample of dendritic cells from the patient;
(b) associating the sample with an agent which binds to a dendritic cell associated protein;
(c) measuring the amount of binding of the agent to the dendritic cell associated protein;
wherein an alteration in the amount of binding is indicative of a disease associated with altered dendritic cell activity.

20. The method of claim 19 wherein the dendritic cells are isolated from a patient suffering from a disease associated with altered dendritic cell activity.

21. The method of claim 19 wherein the disease is an autoimmune disorder or due to an altered immune response.

22. The method of claim 21 wherein the autoimmune disorder is selected from the group consisting of psoriasis, inflammatory bowel disease, asthma, multiple schlerosis, lupus erythematosus, rheumatoid arthritis and type I diabetes.

23. The method of claim 21 wherein the altered immune response is a result of cancer or infectious disease.

24. The method of claim 19 wherein the amount of dendritic cell associated protein expressed is indicative of the extent of the disease.

25. A method of producing an antibody against a dendritic cell associated protein comprising the steps of:
(a) isolating the dendritic cell associated protein;
(b) generating a phage display antibody library against the protein;
(c) associating the phage library with the dendritic cell associated protein; and
(d) isolating an antibody from the phage display antibody library that binds to the protein.

26. The method of claim 25 wherein steps (c) and (d) are repeated to obtain an antibody which binds with higher affinity to the dendritic cell associated protein.

27. The antibody produced by the method of claim 25 or 26.

28. The antibody of claim 27 wherein the antibody is monoclonal.

29. A method of screening for an agent that modulates the expression or activity of a dendritic cell associated protein comprising:
(a) contacting a dendritic cell with a first agent which alters the expression or activity of a protein associated with the dendritic cell;
(b) contacting the dendritic cell with a second agent; and
(c) measuring the level of expression of the dendritic cell associated protein or mRNA encoding the dendritic cell associated protein;
wherein an alteration in the level of expression indicates that the second agent modulates the expression or activity of the dendritic cell associated protein.

30. The method of claim 29 wherein the level of expression is increased following contact with the second agent.

31. The method of claim 29 wherein the level of expression is decreased following contact with the second agent.


SEQUENCE LISTING (10 sequences)

SEQ ID NO: 1 - DNA, 21 bp, Artificial sequence
  PCR Primer Sequence
cctggcaagagcattgtggaa

SEQ ID NO: 2 - DNA, 30 bp, Artificial sequence
  PCR Primer Sequence
tgtaatctatttctttgcttgctctgtcag

SEQ ID NO: 3 - DNA, 24 bp, Artificial sequence
  PCR Primer Sequence
gtgaagagtgagtggtgcaagtat

SEQ ID NO: 4 - DNA, 29 bp, Artificial sequence
  PCR Primer Sequence
cataaagatcccaactagacaaagactgg

SEQ ID NO: 5 - DNA, 24 bp, Artificial sequence
  PCR Primer Sequence
tgggtctgggaacatttctcttca

SEQ ID NO: 6 - DNA, 30 bp, Artificial sequence
  PCR Primer Sequence
ataaatttaatgtacagagtttttggatcc

SEQ ID NO: 7 - DNA, 24 bp, Artificial sequence
  PCR Primer Sequence
ctgagcagtcgtgctggtatcatc

SEQ ID NO: 8 - DNA, 30 bp, Artificial sequence
  PCR Primer Sequence
acccagctgggcaagaaatgcctcaggagg

SEQ ID NO: 9 - DNA, 21 bp, Artificial sequence
  PCR Primer Sequence
gcccagggcaggtgcttatct

SEQ ID NO: 10 - DNA, 29 bp, Artificial sequence
  PCR Primer Sequence
gataacttggtgcaaatacaaagtgaaga